(19) United States
(12) Patent Application Publication
(10) Pub. No.: US 2026/0258498 A1
(43) Pub. Date: Sep. 3, 2026

(54) NON-INVASIVE DIAGNOSTIC TESTS FOR CATTLE EYE DISEASE

(71) Applicant: Board of Supervisors of Louisiana State University and Agricultural and Mechanical College, Baton Rouge, LA (US)

(72) Inventors: Andrew Christopher Lewin, Baton Rouge, LA (US); Chin-Chi Liu, Baton Rouge, LA (US)

(21) Appl. No.: 19/163,877

(22) PCT Filed: Apr. 5, 2024
(86) PCT No.: PCT/US2024/023240

§ 371 Date: Sep. 10, 2025

Related U.S. Application Data
(60) Provisional application No. 63/494,795, filed on Apr. 7, 2023.

(51) Int. Cl. C12Q 1/6883; C12Q 1/6886; C12Q 1/689

(52) U.S. Cl. CPC C12Q 1/6883; C12Q 1/6886; C12Q 1/689

(57) ABSTRACT

Disclosed herein are methods for diagnosing and therefore treating infectious bovine keratoconjunctivitis (IBK) or squamous cell carcinoma (OSCC) in cattle. The methods involve a novel panel of qPCR primer targets, including Bovine GAPDH, Moraxella bovis, Moraxella bovoculi, Mycoplasma, Staphylococcus, Pasteurellaceae, Prevotellaceae, and Weeksellaceae that can be normalized with primers for Universal Bacteria and Escherichia coli. Also provided are coefficients for each of these targets for use in analyzing sample data and predicting IBK or OSCC in cattle.

### CROSS-REFERENCE TO RELATED APPLICATIONS

This application claims benefit of U.S. Provisional Application No. 63/494,795, filed Apr. 7, 2023, which is hereby incorporated herein by reference in its entirety.

### STATEMENT OF GOVERNMENT INTEREST

This invention was made with Government Support under Grant No. 2020-67016-31467 awarded by the United States Department of Agriculture. The Government has certain rights in the invention.

### SEQUENCE LISTING

This application contains a sequence listing filed in ST.26 format entitled “221204-2320 Sequence Listing” created on Apr. 4, 2024, and having 18,477 bytes. The content of the sequence listing is incorporated herein in its entirety.

### BACKGROUND OF THE INVENTION

Ocular squamous cell carcinoma (OSCC) is the most common malignant neoplasia affecting the eyes of cattle, resulting in significant morbidity as well as condemnation at slaughter and economic loss (Tsujita, H, et al. Vet Clin North Am Food Anim Pract 2010, 26:511-529). OSCC manifests as irregular tissue proliferation at the region of the corneoscleral junction, nictitating membrane, cornea, and eyelid (Fornazari, G. A, et al. Vet World 2017, 10:1413-1420). OSCC may metastasize to regional lymph nodes and the lungs, though early excision may be curative (Schulz, K. L, et al. Can Vet J 2010, 51:611-614; Welker, B, et al. Vet Surg 1991, 20:133-139). A microbiome is composed of various microorganisms such as bacteria, fungi, and viruses, co-existing simultaneously in the same environment. The normal bacterial microbiome can be altered in the presence of neoplasia (Mitra, A, et al. Sci Rep 2015, 5:16865; Curty, G, et al. Int J Mol Sci 2019, 21; Turner, N. D, et al. Curr Gastroenterol Rep 2013, 15:346; van Vorstenbosch, R, et al. Metabolites 2022, 13; Wang, Z, et al. Front Microbiol 2020, 11:997; Hashimoto, K, et al. J Oral Microbiol 2022, 14:2105574). The normal and altered bacterial ocular surface microbiome has been characterized in numerous species including cats, dogs, pigs, and horses, among others (Darden, J. E, et al. PLoS One 2019, 14:e0223859; Rogers, C. M, et al. PLoS One 2020, 15:e0234313; Leis, M. L, et al. PLoS One 2021, 16:e0247392; Santibanez, R, et al. Animals (Basel) 2022, 12; Scott, E. M, et al. PLoS One 2019, 14:e0214877). In humans, dysregulation of the normal resident bacterial ocular surface microbiome is associated with several diseases including diabetes, dry eye, and allergic conjunctivitis (Zegans, M. E, et al. Am J Ophthalmol 2014, 158:420-422; Gomes, J. A. P, et al. Asia Pac J Ophthalmol (Phila) 2020, 9:505-511). Understanding bacterial microbiome alterations in ocular disease has potential to inform future diagnostic assays and treatment strategies.

### SUMMARY OF THE INVENTION

Disclosed herein is a method for diagnosing infectious bovine keratoconjunctivitis (IBK) or squamous cell carcinoma (OSCC) in cattle. In some embodiments, the method involves assaying a sample (e.g. conjunctival swab) from the cattle by quantitative PCR (qPCR) using forward and reverse primers specific for a panel of targets comprising Bovine GAPDH, Moraxella bovis, Moraxella bovoculi, Mycoplasma, Staphylococcus, Pasteurellaceae, Prevotellaceae, Weeksellaceae, Universal Bacteria, and Escherichia coli. The method can also involve assaying a known standard of E. coli by quantitative PCR (qPCR) using forward and reverse primers specific for Universal Bacteria and Escherichia coli. The method can then involve calculating average Cycle Threshold (C_{T}) value for each target and normalizing the cattle average C_{T} values using the known standard of E. coli average C_{T} values. The method can finally involve comparing the normalized C_{T} values to control values to predict the presence of IBK or OSCC in the cattle.

In some embodiments, the forward and reverse primers for Bovine GAPDH comprise the nucleic acid sequences SEQ ID NO:1 and 2, respectively; the forward and reverse primers for Moraxella bovis comprise the nucleic acid sequences SEQ ID NO:3 and 4, respectively; the forward and reverse primers for Moraxella bovoculi comprise the nucleic acid sequences SEQ ID NO:5 and 6, respectively; the forward and reverse primers for Mycoplasma comprise the nucleic acid sequences SEQ ID NO:7 and 8, respectively; the forward and reverse primers for Staphylococcus comprise the nucleic acid sequences SEQ ID NO:9 and 10, respectively; the forward and reverse primers for Pasteurellaceae comprise the nucleic acid sequences SEQ ID NO:11 and 12, respectively; the forward and reverse primers for Prevotellaceae comprise the nucleic acid sequences SEQ ID NO:13 and 14, respectively; the forward and reverse primers for Weeksellaceae comprise the nucleic acid sequences SEQ ID NO:15 and 16, respectively; the forward and reverse primers for Universal Bacteria comprise the nucleic acid sequences SEQ ID NO:17 and 18, respectively; the forward and reverse primers for Escherichia coli comprise the nucleic acid sequences SEQ ID NO:19 and 20, respectively, or any combination thereof.

In some embodiments, step e) comprises the use of a data analysis algorithm to weight the C_{T} values according to the coefficients of Table 4.

In some embodiments, the method further involves treating the cattle for IBK or OSCC.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows bacterial microbiome composition of normal and OSCC affected eyes at the phylum level. Eleven different phyla encompassing 120 ASVs were detected on the conjunctival surface of normal and OSCC-affected eyes. OSCC=ocular squamous cell carcinoma.

FIGS. 2A to 2F show alpha diversity of sequenced samples using obs ASV, Chao1 and Faith's PD. Significant differences in alpha diversity indices were observed with respect to geographic location; shown in FIGS. 2A, 2B, and 2C. No significant differences in the same alpha diversity indices was observed with respect to disease status, shown in FIGS. 2D, 2E, and 2F. OSCC=ocular squamous cell carcinoma.

FIGS. 3A and 3B show unweighted unifrac principal coordinates analysis for geographic location (FIG. 3A) and dis-ease status (FIG. 3B). Significance was found between geographic locations (p=0.006) (FIG. 3A) but not by disease status (p=0.211) (FIG. 3B).

FIG. 4 shows relative abundance of bacteria at the phylum level based on 16S rRNA gene sequencing of 228 IBK eyes and 367 Normal eyes (604 samples). The seven most commonly identified phyla encompassing 120 ASVs on the conjunctival surface of IBK and Normal eyes were Actinobacteriota, Bacteroidota, Deferribacterota, Firmicutes, Fusobacteria, Proteobacteria, and Verrucomicrobiota.

FIG. 5A shows overall relative abundance of Actinobacteriota was significantly greater in Normal than IBK eyes (p=0.0040). Three species within Actinobacteriota including Corynebacterium stationis (p=0.0061) (FIG. 5B) and Corynebacterium variabile (p=0.0016) (FIG. 5C) were noted to present at higher relative abundance in Normal eyes than IBK eyes. A third species within Actinobacteriota, Rothia nasimurium (FIG. 5D), was present at higher relative abundance in IBK eyes (p<0.0001). Student's t tests or ANOVA tests were used to evaluate abundance.

FIG. 6 shows the overall abundance of the Moraxella genus was significantly higher in IBK eyes compared with Normal eyes (p<0.0001). Student's t tests or ANOVA tests were used to evaluate differences in relative abundance.

FIGS. 7A to 7F show observed ASV against disease status (FIG. 7A), sex (FIG. 7B), US state (FIG. 7C), farm (FIG. 7D), age (FIG. 7E), and breed purpose (FIG. 7F) evaluated with non-parametric Kruskal-Wallis and Mann-Whitney U tests. No significant differences in alpha-diversity with regard to disease status were observed with Obs ASV (p>0.05). Significant differences in alpha-diversity were detected with regard to geographic location (both by state and farm, p<0.0001), age (p<0.0001), sex (p=0.0379), and breed (p<0.0001) for Obs ASV.

FIGS. 8A to 8D show unweighted unifrac principal coordinate analysis performed by PERMANOVA of disease status (IBK or Normal) (FIG. 8A), geographic location (state) (FIG. 8B), sex (FIG. 8C), and breed (beef and dairy) (FIG. 8D). There were significant differences in beta-diversity between IBK and Normal eyes (p=0.002), between geographic locations (state) (p=0.001), between male and female cattle (p=0.001), and between beef and dairy cattle (p<0.001). IBK=infectious bovine keratoconjunctivitis eyes, Normal=normal eyes, F=Female, M=Male.

FIG. 9 shows column contribution of each primer in random forest algorithms presented as relative deviance (G²). The three most consistent canonical variables to classify disease status through data modeling of RT-PCR included Moraxella bovoculi, Moraxella bovis, and Staphylococcus spp. The three least significant primers contributing to the canonical variable in the primer set were Pasteurellaceae, Mycoplasma, and Prevotellaceae.

FIG. 10 is a flow chart of the experimental design for evaluation of the bovine bacterial OSM, including initial ophthalmic examination, diagnosis of active IBK, inactive IBK, or normal eyes, and the specific subsequent samples collected for further analysis.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “Cycle Threshold” or “C_{T}” as used herein refers to the number of amplification cycles required for the fluorescent signal to exceed the basal threshold level. In a real time PCR assay a positive reaction is detected by accumulation of a fluorescent signal. The C_{T} (cycle threshold) is defined as the number of cycles required for the fluorescent signal to cross the threshold (i.e. exceeds background level). C_{T} levels are inversely proportional to the amount of target nucleic acid in the sample (i.e. the lower the C_{T} level the greater the amount of target nucleic acid in the sample).

### Predictive Analysis

In some embodiments, step e) of the disclosed method comprises the use of a data analysis algorithm to weight the C_{T} values according to the coefficients of Table 4.

The analytic classification process may be any type of learning algorithm with defined parameters, or in other words, a predictive model. In general, the analytical process will be in the form of a model generated by a statistical analytical method such as those described below. Examples of such analytical processes may include a linear algorithm, a quadratic algorithm, a polynomial algorithm, a decision tree algorithm, or a voting algorithm.

Using any suitable learning algorithm, an appropriate reference or training dataset can be used to determine the parameters of the analytical process to be used for classification, i.e., develop a predictive model. The reference or training dataset to be used will depend on the desired classification to be determined. The dataset may include data from two, three, four or more classes.

The number of features that may be used by an analytical process to classify a test subject with adequate certainty is 2 or more. In some embodiments, it is 3 or more, 4 or more, 10 or more, or between 10 and 200. Depending on the degree of certainty sought, however, the number of features used in an analytical process can be more or less, but in all cases is at least 2. In one embodiment, the number of features that may be used by an analytical process to classify a test subject is optimized to allow a classification of a test subject with high certainty.

Suitable data analysis algorithms are known in the art. In one embodiment, a data analysis algorithm of the disclosure comprises Classification and Regression Tree (CART), Multiple Additive Regression Tree (MART), Prediction Analysis for Microarrays (PAM), or Random Forest analysis. Such algorithms classify complex spectra from biological materials, such as a blood sample, to distinguish subjects as normal or as possessing biomarker levels characteristic of a particular condition (e.g., @@@). In other embodiments, a data analysis algorithm of the disclosure comprises ANOVA and nonparametric equivalents, linear discriminant analysis, logistic regression analysis, nearest neighbor classifier analysis, neural networks, principal component analysis, hierarchical cluster analysis, quadratic discriminant analysis, regression classifiers and support vector machines.

As will be appreciated by those of skill in the art, a number of quantitative criteria can be used to communicate the performance of the comparisons made between a test marker profile and reference marker profiles. These include area under the curve (AUC), hazard ratio (HR), relative risk (RR), reclassification, positive predictive value (PPV), negative predictive value (NPV), accuracy, sensitivity and specificity, Net reclassification Index, Clinical Net reclassification Index. In addition, other constructs such a receiver operator curves (ROC) can be used to evaluate analytical process performance.

### Method of Treating IBK

In some embodiments, the method further involves treating the cattle for IBK. Although Moraxella bovis has long been considered to be the etiologic agent of IBK, other species of Moraxella, such as M. bovoculi, M. ovis, various Mycoplasma species (M. bovis and M. bovoculi), and infection with bovine herpesvirus type 1 and infectious bovine rhinotracheitis (IBR) virus may also play a role in disease.

Moraxella spp are susceptible to many antibiotics. Because antibiotic susceptibility may vary in different geographic locations, susceptibility testing of isolated organisms is advised. In the USA, long-acting oxytetracycline (two injections of 20 mg/kg, IM or SC, at a 48- to 72-hour interval) and tulathromycin (2.5 mg/kg, SC, given once) are approved for IBK treatment in cattle. Other effective (but not FDA-approved) antibiotics include ceftiofur crystalline free acid (6.6 mg/kg, SC, at the base of the ear) and florfenicol (20 mg/kg, IM, two doses at a 2-day interval). According to current federal regulations in the USA, use of ceftiofur in cattle must follow approved bottle directions, including dose, route, and frequency. Another common treatment for IBK is bulbar conjunctival injection with penicillin.

For infectious keratitis in sheep and goats, topical oxytetracycline and antiseptic sprays are currently approved treatments. Topical applications should be applied at least three times a day to be effective, and thus are often not cost-effective or practical in herd settings.

A third-eyelid flap or partial tarsorrhaphy, which will shade the cornea from sunlight, together with subconjunctival injection, may reduce morbidity in severely affected animals. A temporary eye patch glued to the hair surrounding the eye is an inexpensive and easily applied treatment. The eye patch provides shade, prevents exposure to flies, and may help to decrease spread of organisms.

Animals with substantial uveitis secondary to keratoconjunctivitis that is particularly painful may benefit from topical ophthalmic application of 1% atropine ointment 1-3 times daily. This will prevent painful ciliary body spasms and reduce the likelihood of posterior synechia formation that occurs with miosis. Because of mydriasis caused by atropine, treated animals should be provided with shade. Systemic NSAID treatment (eg, flunixin meglumine) may also provide relief.

### Method of Treating OSCC

In some embodiments, the method further involves treating the cattle for OSCC. Treatment is highly dependent on the location of the tumor and the degree of invasion of the underlying tissue. Some surgical procedures include eyelid wedge resection, third eyelid resection, or enucleation of the entire globe and lid margins. Surgical treatment methods do not always mean a cure for the disease. A 40-50% recurrence rate can be expected using these methods.

If the cancer has spread to regional lymph nodes, these surgical methods will not cure the disease and the tumor will continue to grow at those sites. If the tumor has not invaded underlying and adjacent tissues cryosurgery is a useful therapeutic tool. Cryosurgery is a means by which a tumor can be frozen off. This method works well on small tumors (<1″ diameter) that have not invaded underlying tissues. Cryosurgery commonly leaves a scar on the surface of the eye that may interfere with future vision. Using cryosurgical methods, tumor removal has a high success rate (90%). Eyelid tumors are somewhat less successfully removed by these means (60%).

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Relative and Quantitative Characterization of the Bovine Bacterial Ocular Surface Microbiome in the Context of Ocular Squamous Cell Carcinoma

### Introduction

Bacterial relative abundance is frequently assessed using 16S ribosomal ribonucleic acid (rRNA) gene sequencing, which utilizes bacterial conserved and hypervariable regions to classify bacteria within a sample into operational taxonomic units (OTUs) or amplicon sequence variants (ASVs) (Chiarello, M, et al. PLOS ONE 2022, 17:e0264443; Janda, J. M, et al. J Clin Microbiol 2007, 45:2761-2764; Mignard, S, et al. J Microbiol Methods 2006, 67:574-581). The taxonomic diversity of ASVs within a sample is evaluated for richness and evenness of distribution using different indices, such as Observed (Obs) ASV, Simpson, Shannon, Chao1, Pielou, and Faith's phylogenetic diversity (PD), to measure a sample's alpha-diversity (Banks, K. C, et al. Vet Ophthalmol 2019, 22:716-725). The taxonomic diversity of ASVs between samples (beta-diversity) is evaluated through various pairwise distance metrics such as weighted and unweighted unifrac analysis (Banks, K. C, et al. Vet Ophthalmol 2019, 22:716-725). The ocular surface is a relatively low biomass environment and methods routinely utilized for 16S rRNA gene sequencing relative abundance analysis require various modifications to be suitable for this application (Banks, K. C, et al. Vet Ophthalmol 2019, 22:716-725; Suchodolski, J. S. Vet J 2016, 215:30-37; Scott, E. M, et al. Vet Ophthalmol 2021, 24:4-11).

For absolute characterization of selected elements of the bacterial ocular microbiome at the species level and higher, real-time polymerase chain reaction (RT-PCR) can be utilized (Sung, C. H, et al. J Feline Med Surg 2022, 24:e1-e12; AlShawaqfeh, M. K, et al. FEMS Microbiol Ecol 2017, 93). In RT-PCR, the number of cycles required to amplify a signal above a specified threshold value (C_{T}) value allows for estimation of bacterial load. Primers targeting several bacterial families, genera or species can be simultaneously assessed using RT-PCR (Sung, C. H, et al. J Feline Med Surg 2022, 24:e1-e12; AlShawaqfeh, M. K, et al. FEMS Microbiol Ecol 2017, 93; Bartenslager, A. C, et al. Animal Microbiome 2021, 3). These bacterial targets can be chosen based on previous clinical information or relative abundance analysis. While primer specificity limits the number of bacterial species detected compared with 16S rRNA gene sequencing, estimation of bacterial quantity provides unique meaningful insights which otherwise cannot be gained (Suchodolski, J. S. Vet J 2016, 215:30-37; Sung, C. H, et al. J Feline Med Surg 2022, 24:e1-e12; AlShawaqfeh, M. K, et al. FEMS Microbiol Ecol 2017, 93; Suchodolski, J. S. Vet Clin Pathol 2022, 50 Suppl 1:6-17).

The bovine ocular surface microbiome (BBOSM) has been previously investigated in calves (Bartenslager, A. C, et al. Animal Microbiome 2021, 3; Cullen, J. N, et al. Vet Microbiol 2017, 207:267-279). In one previous study which utilized 16S rRNA gene sequencing analysis, the relative abundance of Weeksellaceae, Methylobacteriaceae, and Mycoplasmataceae were found to be significantly different on the conjunctival surface of eyes with and without infectious bovine keratoconjunctivitis (IBK) (Cullen, J. N, et al. Vet Microbiol 2017, 207:267-279). In addition, clustering of samples based on geographic grouping of animals was observed (Cullen, J. N, et al. Vet Microbiol 2017, 207:267-279). A second study used similar methods in an attempt to predict which animals which will go on to develop IBK (Bartenslager, A. C, et al. Animal Microbiome 2021, 3). Pasteurellaeceae, Moraxella, Mycoplasma, and Weeksellaceae were among the most common families and genera detected in normal cattle in this study (Bartenslager, A. C, et al. Animal Microbiome 2021, 3).

The BBOSM has not yet been studied in the presence of OSCC. This study aimed to assess the BBOSM using both a relative abundance (16S rRNA gene sequencing analysis) and absolute abundance (RT-PCR) approach. It was hypothesized that there would be both relative and absolute abundance differences in the BBOSM in normal and OSCC-affected eyes (Bartenslager, A. C, et al. Animal Microbiome 2021, 3).

### Materials and Methods

### Study Population and Sample Collection

Nineteen cows (38 eyes) from two different geographic locations (Louisiana and Wyoming) were included in the study.

A gross ophthalmic examination of all animals was performed by a trained veterinary observer (HBG). Lesions consistent with OSCC including plaques, keratosis, papillomas, non-invasive carcinomas, and invasive carcinomas and the lesion location (eyelid, nictitating membrane, limbus) were identified and documented. For all animals, both lower conjunctival fornices were individually sampled by one observer (HBG) with twin swabs (Isohelix DNA Buccal Swab Pack, MidSCi, St Louis, MO) by vigorously rubbing the conjunctiva for 2-5 seconds. Swabs were placed in labeled 15 mL sterile centrifuge tubes (VWR, Radnor, PA) prior to being stored on ice. Non-sterile gloves (VWR, Radnor, PA) were worn and changed between each animal during conjunctival swab sample collection. Where possible, samples from normal animals were collected from the same geographic location as OSCC affected animals. Environmental control samples were collected at each geographic location by exposing twin swabs to the air and immediately storing these in a 15 mL tube on ice.

### Confirmation of OSCC

For confirmation of OSCC in affected animals, an incisional biopsy was collected from eyelid and nictitating membrane lesions where possible, following swab collection as outlined above. The small size of some lesions precluded safe collection of a confirmatory biopsy; specimens were obtained from 6/10 eyes exhibiting lesions consistent with OSCC. Proparacaine hydrochloride ophthalmic solution (0.5%, Akorn, Inc., Lake Forest, IL) was applied to the ocular surface followed by povidone-iodine solution (AVRIO Health, Stamford, CT). A regional infiltrative block of 2% lidocaine (VetOne, MWI Animal Health, Boise, ID) was injected prior to removal of eyelid lesions. Incisional biopsy was performed using a #10 or #15 scalpel blade, or scissors. The biopsy specimen was placed in a 10% neutral buffered formalin container for fixation and storage. Biopsy samples fixed in 10% formalin for a minimum of 1 month were frozen in paraffin and cut in 5 μm serial sections, then stained with hematoxylin and eosin for histopathological analysis by a residency-trained veterinary anatomic pathologist (LPG).

### DNA Extraction

DNA Extraction was performed using the DNeasy PowerSoil Pro Kit (QIAGEN GmbH, Hilden, Germany) by following the manufacturer's instructions. DNA was extracted from each conjunctival swab and from the environmental control samples, as well as from extraction control samples created by replicating the protocol in the absence of swabs. Extractions were performed in a filtered laminar flow cabinet (The Clone Zone, USA/Scientific, Inc., Ocala, Florida, USA). Concentrations of each eluted DNA sample were calculated (NanoDrop One Microvolume UV-Vis Spectrophotometer, ThermoFisher Scientific, Waltham, MA) and documented prior to storage at −80° C.

16S rRNA Gene Sequencing and Analysis

DNA sequencing was performed by the Louisiana State University School of Medicine Microbial Genomics Resource Group. Two steps of amplification were performed to prepare the sequencing library using the AccuPrime Taq high fidelity DNA polymerase system (Invitrogen, Carlsbad, CA). Negative controls (DNA extraction and environment) and a positive control (Microbial mock community HM-276D, BEI Resources, Manassas, VA) were also processed during amplicon library preparation. The hypervariable V4 region was amplified using 20 ng genomic DNA and gene-specific primers with Illumina adaptors. The PCR conditions were as follows: 95° C. for 3 min, 25 cycles of 95° C. for 30 s, 55° C. for 30 s and 72° C. for 30 s, 72° C. for 5 min and holding at 4° C. PCR products were purified using AMPure XP beads with beads added as 0.85× the PCR volume. Four μL of purified amplicon DNA from the previous step was amplified for 8 cycles with the same PCR conditions using primers with different molecular barcodes. The indexed amplicon libraries were purified using AMPure XP beads and quantified using Quant-iT PicoGreen (Invitrogen), then normalized and pooled. The pooled library was quantified using the KAPA Library Quantification Kit (Kapa Biosystems, Cape Town, South Africa), diluted and denatured as per Illumina guidelines. Ten percent Illumina PhiX was added to the sequencing library as an internal control and to increase diversity of the 16S rRNA amplicon library. Paired-end sequencing was performed using an Illumina MiSeq (Illumina, San Diego, CA) using the 2×250 bp V2 sequencing kit. The sequencing reads were transferred to Illumina's BaseSpace for quality analysis, and the generated raw FASTQ files were used for further bioinformatics analysis.

Sequencing reads from FASTQ files were imported into R version 4.2.0 and processed with DADA2 (Callahan, B. J, et al. Nat Methods 2016, 13:581-583) version 1.22.0. Read quality profiles were examined to select appropriate trimming and filtering parameters which were set to truncate reads to 240 bp (both forward and reverse) to remove low quality tails, and trim 20 bp (left) of each read. The standard DADA2 workflow was utilized, including error learning and sample inference for forward and reverse reads followed by merging of sequence variants. Sequence variants outside of the expected amplicon size of 249 to 256 bp were removed and chimeric sequence variants were detected and removed using ‘removeBimeraDenovo’. Remaining sequence variants were placed into a sequence table with read counts ranging from 816 to 143459. Taxonomy was classified using the SILVA database (Quast, C, et al. Nucleic Acids Res 2013, 41:D590-596) and mapping information was imported to construct a Phyloseq (McMurdie, P. J, et al. PLoS One 2013, 8:e61217) object. Downstream analysis was performed using Phyloseq (McMurdie, P. J, et al. PLoS One 2013, 8:e61217) version 1.38.0. Suspected contaminant ASV were identified and removed using Decontam (Davis, N. M, et al. Microbiome 2018, 6:226) version 1.14.0 with the prevalence method. An abundance filter was used to filter out remaining ASVs with a mean of less than 104 across all samples.

### RT-PCR Processing and Analysis

RT-PCR for selected bacterial genera, families and species (Table 1) was performed using remaining eluted DNA and PerfeCTa® SYBR® Green FastMix®, ROX™ (VWR, Radnor, PA). Primer pairs for bacterial targets were chosen based on previous clinical reports and exploratory 16S rRNA gene sequencing relative abundance analyses obtained from cattle with eye disease (Bartenslager, A. C, et al. Animal Microbiome 2021, 3; Cullen, J. N, et al. Vet Microbiol 2017, 207:267-279). Eluted DNA was diluted with molecular grade water (VWR, Radnor, PA) to concentrations standardized between eyes of the same cow with an overall range of 0.3 to 34.3 ng/μl. Each DNA sample was individually combined in triplicate with nine different primer pairs. The RT-PCR panel performed on each sample included the following primer (IDT, Coralville, IA) targets: bovine GAPDH (Menzies, M, et al. Vet Immunol Immunopathol 2006, 109:23-30), Moraxella bovis (Zheng, W, et al. J Microbiol Methods 2019, 160:87-92), Moraxella bovoculi (Zheng, W, et al. J Microbiol Methods 2019, 160:87-92), Mycoplasma (van Kuppeveld, F. J, et al. Appl Environ Microbiol 1992, 58:2606-2615), Pasteurellaceae (Bootz, F, et al. Lab Anim Sci 1998, 48:542-546), Prevotellaceae (Stevenson, D. M, et al. Appl Microbiol Biotechnol 2007, 75:165-174), Staphylococcus (Morot-Bizot, S. C, et al. J Appl Microbiol 2004, 97:1087-1094), Weeksellaceae and ‘universal bacteria’ (AlShawaqfeh, M. K, et al. FEMS Microbiol Ecol 2017, 93). Bovine GAPDH primer was utilized as a reference for host DNA in proportion to the total bacterial DNA extracted, while universal bacteria primer was used for data normalization. Molecular grade water (VWR, Radnor, PA) and Escherichia coli standard (10 ng/μL, Sigma-Aldrich, Saint Louis, MO) were used as negative and positive controls, respectively. The E. coli standard was plated in triplicate with both ‘universal bacteria’ primers and E. coli primers (Sidjabat, H. E, et al. J Antimicrob Chemother 2006, 57:840-848) to allow for standardization between runs and relative quantification of non-E. coli bacterial groups. The RT-PCR run included a hold stage of 50° C. for 2 minutes and 95° C. for 10 minutes; a PCR stage of 40 cycles of 95° C. for 15 seconds followed by 60° C. for 10 minutes; a continuous melt curve stage of 95° C. for 15 seconds, 60° C. for 15 seconds; and a dissociation step of 95° C. for 15 seconds. The RT-PCR data was expressed as the log amount of DNA (ag) for each primer pair per 10 ng of isolate total DNA using an E. coli standard on the same RT-PCR plate. The Ct values of each primer set were normalized by universal bacteria Ct values within each sample for further analyses.

TABLE 1
Primers used for the RT-PCR panel. Please note that the ‘Universal Bacteria’
and the Escherichia coli primer sets were used for normalization purposes
and these values were not included in the discriminant analyses models.

| Target | Forward primer | Reverse primer |
|---|---|---|
| Bovine GAPDH | CCTGGAGAAACCTGCCAAGT (SEQ ID NO: 1) | GCCAAATTCATTGTCGTACCA (SEQ ID NO: 2) |
| Moraxella bovis | GGTGACGACCGCTTGTTT (SEQ ID NO: 3) | ATCATCGCCTTCATCTCCAG (SEQ ID NO: 4) |
| Moraxella bovoculi | GGTGATATTTATCATGAAGTTGTGAAA (SEQ ID | TCTCAATTCATAATCACGATACTCAAG (SEQ ID |
| | NO: 5) | NO: 6) |
| Mycoplasma | TGCACCATCTGTCACTCTGTTAACCTC (SEQ ID | ACTCCTACGGGAGGCAGCAGTA (SEQ ID NO: 8) |
| | NO: 7) | |
| Staphylococcus | GGCCGTGTTGAACGTGGTCAAATCA (SEQ ID | ACCATTTCAGTACCTTCTGGTAA (SEQ ID NO: 10) |
| | NO: 9) | |
| Pasteurellaceae | CATAAGATGAGCCCAAG (SEQ ID NO: 11) | GTCAGTACATTCCCAAGG (SEQ ID NO: 12) |
| Prevotellaceae | GGTTCTGAGAGGAAGGTCCCC (SEQ ID NO: 13) | TCCTGCACGCTACTTGGCTG (SEQ ID NO: 14) |
| Weeksellaceae | ATCCAGCCATCCCGCGT (SEQ ID NO: 15) | CTGCTGGCACGGAGTTAGC (SEQ ID NO: 16) |
| Universal Bacteria | CCTACGGGAGGCAGCAGT (SEQ ID NO: 17) | ATTACCGCGGCTGCTGG (SEQ ID NO: 18) |
| Escherichia coli | CCGATACGCTGCCAATCAGT (SEQ ID NO: 19) | ACGCAGACCGTAGGCCAGAT (SEQ ID NO: 20) |

### Statistical Analysis

All statistical analyses were performed using commercial software (JMP Pro and R Statistical Software v4.1.3). Associations between eye sampled (right or left), geographic location, age, and OSCC status were checked via chi-squared test. Abundance, alpha diversity from 16S rRNA gene sequencing, Log DNA concentration from RT-PCR were analyzed with T test or Mann-Whitney tests against disease status or location. For beta diversity, standard weighted unifrac analysis, unweighted unifrac analysis, and Bray-Curtis analysis were evaluated via permutational multivariate analysis of variance (PERMANOVA) using vegan R package 2.6.2. Quadratic discriminant analyses (QDA) were used to categorize combined RT-PCR data (normalized C_{T} values for 8 primer sets) by disease status (normal or OSCC). Linear discriminant analysis (LDA) was used to categorize combined RT-PCR data by geographic location (Louisiana or Wyoming). Two training sets of 30 random samples were used to generate the QDA model. A validation set of 8 samples was randomly generated via sample function in R for normal and OSCC samples. For both the QDA and LDA models, primer pairs were removed individually to evaluate the relative contribution of each primer to the model. Statistical significance was at p<0.05.

### Results

### Sampling Demographics

38 eyes (28 normal, 10 OSCC) from 19 cows (all adult females; 10 unaffected, 9 with unilateral or bilateral OSCC lesions) were included in this study. The median age of cows sampled was 6 years (range 2-13 years). Nine animals were between the ages of 1 years and 5 years, while 10 cows were 6 years or older. Increasing age (6 years or older) was significantly associated with a higher likelihood of OSCC disease status (p=0.0376). Samples were taken from one cow with unilateral OSCC lesions in Louisiana; all other samples were taken from cattle at a single farm in Wyoming. One environmental negative control sample was obtained at each geographic location.

Eighteen Hereford cattle and 1 Hereford crossed with Red Angus were sampled. Nine cattle had lesions consistent with OSCC: 8 cows had unilateral ocular lesions, while 1 cow had bilateral ocular lesions consistent with OSCC. Two OSCC lesions were located on the nictitating membrane only, 3/10 OSCC lesions were associated with the corneoscleral junction of the globe, 3/10 OSCC lesions were associated with the eyelids, and 2/10 eyes had lesions on both the upper eyelid and nictitating membrane. Histopathology of 6/6 lesions biopsied confirmed OSCC. Histologic alterations characteristic of OSCC were noted in all samples, including neoplastic polygonal squamous epithelial cells arranged in anastomosing trabeculae, and islands with moderate to marked anisocytosis and anisokaryosis as well as variable dyskeratosis and keratinization of neoplastic cells (keratin pearls).

16S rRNA Gene Sequencing

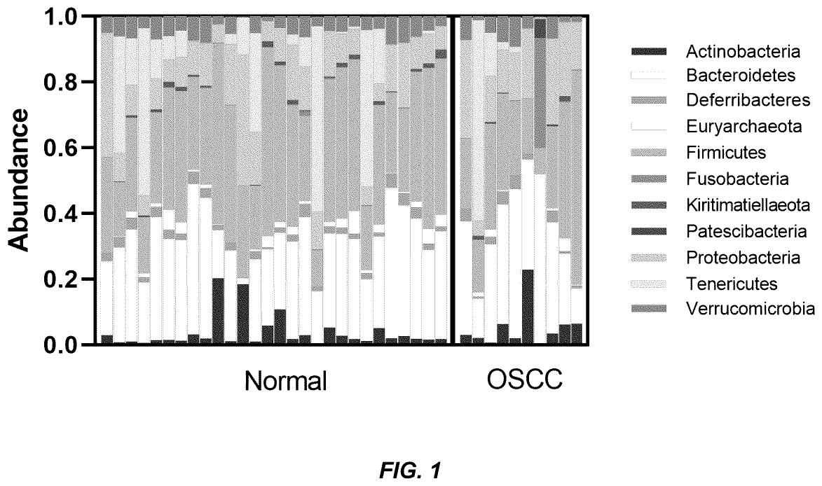

Bacterial population composition. From 41 total samples (38 conjunctival swabs, two environmental swabs, 1 sequencing control), 9768 ASVs were sequenced. With potential contaminants (ASVs encompassing less than 0.001% frequency) removed, 1079 ASVs were sequenced. An even sequencing depth of 120 ASVs for the 38 samples were then graphed (FIG. 1). The 11 most abundant phyla, encompassing 120 ASVs, included Actinobacteria, Bacteroidetes, Deferribacteres, Euryarchaeota, Firmicutes, Fusobacteria, Kiritmatiellaeota, Patescibacteria, Proteobacteria, Tenericutes, and Verrucomicrobia. The three most abundant phyla recorded within both the control and OSCC populations included Bacteroidetes, Firmicutes, and Proteobacteria (Table 2). Among the 11 most abundant phyla, only Euryarchaeota was found at a significantly lower relative abundance in OSCC eyes compared to normal eyes (p=0.0372). The relative abundance of Euryarchaeota, Kiritimatiellaeota, and Proteobacteria were significantly different between two geographic locations (p=0.0057) (Table 2).

TABLE 2
Summary of 16S rRNA gene sequencing results at the phylum level.

| | Median (Min-Max) Abundance Composition (%) | | | | | | | |
|---|---|---|---|---|---|---|---|---|
| | Disease status | | | | Location | | | |
| | Normal | | OSCC | | Louisiana | | Wyoming | |
| Phylum | (n = 28) | | (n = 10) | | (n = 2) | | (n = 36) | |
| Actinobacteria | 1.75 | (0.49-20.17) | 3.19 | (0.25-22.85) | 2.92 | (2.86-2.98) | 1.90 | (0.25-22.85) |
| Bacteroidetes | 29.27 | (1.88-45.95) | 33.69 | (10.70-51.72) | 28.62 | (22.61-34.62) | 30.23 | (1.88-51.72) |
| Deferribacteres | 2.08 | (0.16-4.29) | 1.87 | (0.06-4.23) | 3.25 | (2.55-3.95) | 2.10 | (0.06-4.29) |
| Euryarchaeota | 1.26 | (0.02-6.90)* | 0.40 | (0.00-3.81)* | 0.01 | (0.00-0.02)* | 1.15 | (0.01-6.90)* |
| Firmicutes | 32.88 | (10.76-57.51) | 23.69 | (7.89-65.20) | 25.21 | (21.27-29.15) | 29.35 | (7.89-65.20) |
| Fusobacteria | 0.04 | (0.00-1.46) | 0.01 | (0.00-33.53) | 0.00 | (0.00-0.00) | 0.03 | (0.00-33.53) |
| Kiritimatiellaeota | 0.60 | (0.00-2.61) | 0.23 | (0.00-1.51) | 0.00 | (0.00-0.00)* | 0.52 | (0.00-2.61)* |
| Patescibacteria | 0.00 | (0.00-0.06) | 0.00 | (0.00-5.70) | 0.00 | (0.00-0.00) | 0.00 | (0.00-5.70) |
| Proteobacteria | 9.56 | (5.39-39.85) | 14.58 | (0.59-29.80) | 33.75 | (29.80-37.71)* | 10.07 | (0.59-39.85)* |
| Tenericutes | 1.97 | (0.06-56.49) | 0.20 | (0.02-60.99) | 0.16 | (0.16-0.17) | 1.36 | (0.02-60.99) |
| Verrucomicrobia | 4.85 | (0.38-8.59) | 4.38 | (0.15-9.26) | 6.08 | (4.94-7.21) | 4.62 | (0.15-9.26) |

An asterisk (*) denotes significant differences in % abundance by disease status or location.
OSCC = ocular squamous cell carcinoma.

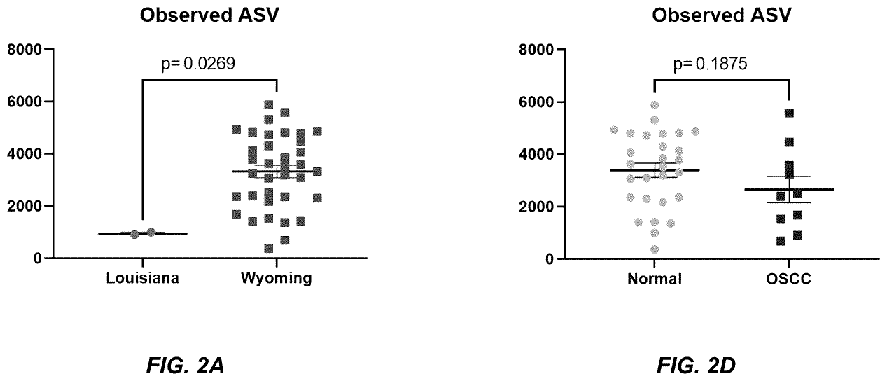

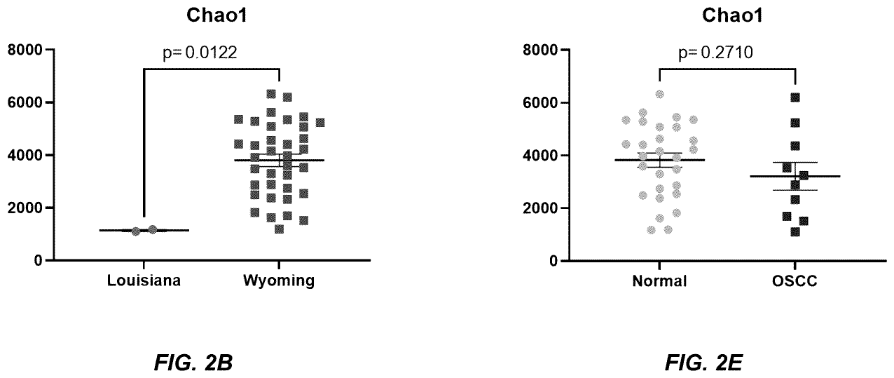

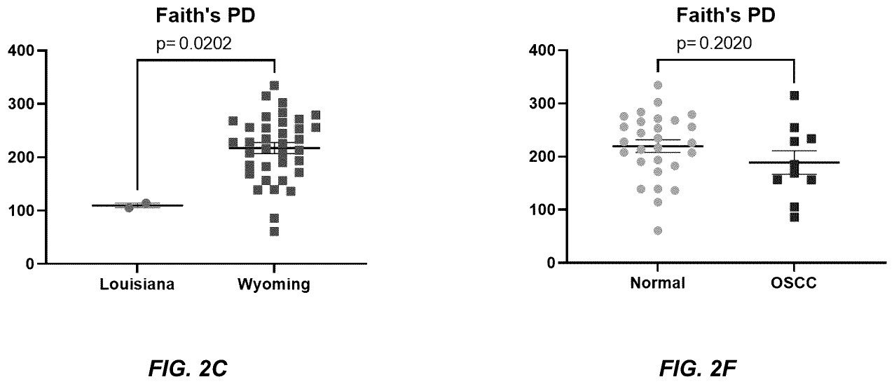

Alpha diversity analysis. Indices chosen to evaluate the alpha diversity of samples included Obs ASV (richness of microbial communities present (Scott, E. M, et al. PLoS One 2019, 14:e0214877)), Chao1 (richness at full sequence depth (Scott, E. M, et al. PLoS One 2019, 14:e0214877)), and Faith's PD (the sum of the branch lengths of phylogenetic tree connecting each species (Chao, A, et al. Methods in Ecology and Evolution 2015, 6:380-388)). Significance differences in alpha diversity were detected based on geographic location using Obs ASV (p=0.0239), Chao1 (p=0.0122), and Faith's PD (p=0.0202) (FIG. 2). This finding was not unexpected due to the small sample size at one location (Louisiana: n=1 cow, 2 eyes) compared to the second location (Wyoming: n=18 cows, 36 eyes). Significant difference in sample alpha diversity was not attributed to other variables described, including left vs. right eye, age of animal, breed, or disease status (normal or OSCC) (FIG. 2).

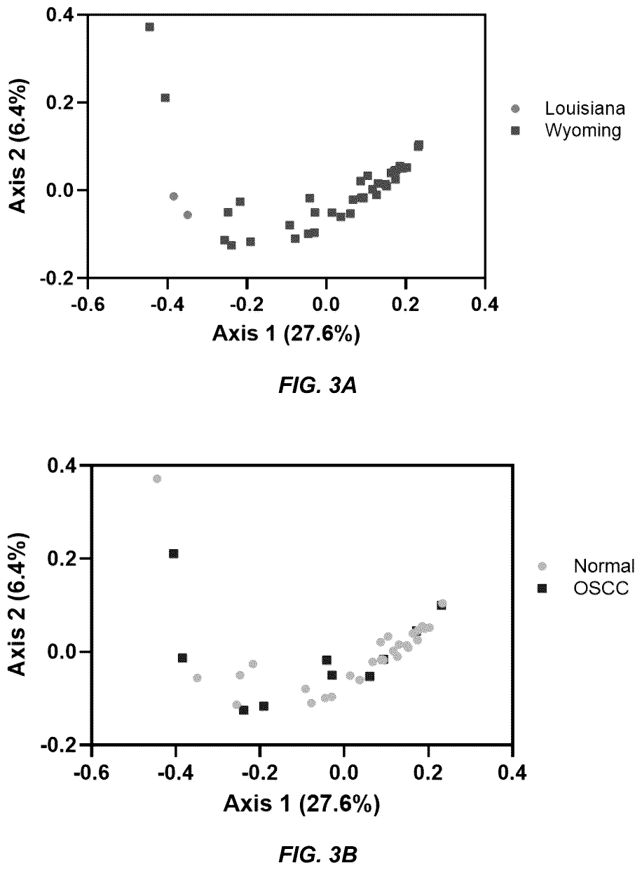

Beta diversity analysis. Beta diversity was evaluated using standard weighted unifrac analysis, unweighted unifrac analysis, and Bray-Curtis analysis. Comparisons were made between left vs right eyes, geographic location (Louisiana or Wyoming), age (1 to 5 years or over 6 years), breed (Hereford or Hereford cross, and disease status (normal or OSCC). Significant differences between geographic locations (p=0.006) (FIG. 3) were found only using unweighted unifrac analysis, a finding once again not unexpected due to the disparity in sample sizes between locations. Significant differences in beta diversity were not attributed to other variables, including disease status (p=0.211) (FIG. 3).

### RT-PCR Analysis

RT-PCR results. A total of 38 DNA samples were prepared and processed using RT-PCR with normalized results shown in Table 3. The quantity of Pasteurellaceae was significantly higher in OSCC-affected eyes compared to normal eyes (p=0.0279). When comparing geographic locations, Moraxella bovis, Mycoplasma and Prevotellaceae were found in significantly lower quantities in samples from Louisiana compared to Wyoming (p=0.0341, 0.0057, 0.0014, respectively) (Table 3).

Quadratic and linear discriminant analyses (QDA/LDA). The C_{T} values of eight primer sets (Table 1) were normalized by the universal bacterial primers for the same sample. The data was further analyzed by discriminant analysis for disease status (normal or OSCC) and geographic location (Louisiana or Wyoming). Due to the unbalanced sample size at each location, only 30 samples were used to construct the canonical variable table. Two randomized validation sets (n=8) were used for disease status. The standardized canonical coefficients from two different training sets for disease status and one for geographic location are listed in Table 4.

TABLE 3
Summary of normalized RT-PCR results. The values shown are
the log amount of DNA (ag) per 10 ng of isolated total DNA.

| | Median (Min-Max) Log DNA (ag) per 10 ng isolated DNA | | | |
|---|---|---|---|---|
| | Disease status | | Location | |
| Target primer | Normal (n = 28) | OSCC (n = 10) | Louisiana (n = 2) | Wyoming (n = 36) |
| Bov. GAPDH | 7.39 (7.11-7.59) | 7.46 (6.96-7.57) | 7.44 (7.42-7.47) | 7.39 (6.96-7.59) |
| Moraxella bovis | 3.45 (0.00-4.87) | 3.25 (2.42-4.26) | 2.71 (2.42-3.00)* | 3.38 (0.00-4.87)* |
| Moraxella bovoculi | 3.11 (0.00-5.75) | 3.50 (0.00-6.43) | 3.83 (2.27-5.38) | 3.11 (0.00-6.43) |
| Mycoplasma | 6.07 (3.54-8.06) | 5.08 (0.00-8.42) | 1.79 (0.00-3.58)* | 5.98 (3.46-8.42)* |
| Staphylococcus | 4.13 (0.00-7.74) | 5.02 (0.00-7.46) | 5.62 (5.24-6.00) | 4.19 (0.00-7.74) |
| Pasteurellaceae | 4.27 (3.72-7.22)* | 5.63 (3.69-7.10)* | 5.09 (4.51-5.66) | 4.34 (3.69-7.22) |
| Prevotellaceae | 7.00 (5.55-7.85) | 8.82 (4.97-8.98) | 5.26 (4.97-5.55)* | 7.00(5.85-8.99)* |
| Weeksellaceae | 6.09 (5.09-7.72) | 6.22 (5.19-7.10) | 5.56 (5.19-5.93) | 6.20 (5.09-7.72) |

An asterisk (*) denotes significant differences in % abundance by disease status or location.
OSCC = ocular squamous cell carcinoma.

TABLE 4
Standardized canonical coefficients and performance
of quadratic and linear discriminant analysis.

| Disease status (n = 30) | | Location |
|---|---|---|
| Training Set #1 | Training Set #2 | (n = 38) |

Canonical standardized coefficients

| | | | |
|---|---|---|---|
| Bov. GAPDH | 0.1824 | 0.3792 | 0.3741 |
| Moraxella bovis | −0.4893 | 0.1722 | 0.2181 |
| Moraxella bovoculi | 0.1398 | 0.2992 | 0.2206 |
| Mycoplasma | 0.4204 | 0.2334 | −0.7565 |
| Staphylococcus | 0.5929 | −0.3859 | −0.6343 |
| Pasteurellaceae | −0.9643 | 0.9771 | 0.1322 |
| Prevotellaceae | 0.0860 | 0.3149 | −0.5426 |
| Weeksellaceae | 0.3759 | 0.7158 | 0.2818 |
| Sensitivity (%) | 100^{a}, 100^{b} | 100^{a}, 100^{b} | |
| Specificity (%) | 95.5^{a}, 83.3^{b} | 100^{a}, 100^{b} | |
| Misclassified (%) | | | 2.6 |

^{a}= values from training set.
^{b}= values from validation set.

Quadratic discriminant analyses (QDA) were used to separate disease status (OSCC or normal) using a training set composed of 30 samples (Table 4). The QDA generated from the training set performed well on the validation sets, resulting in 100% sensitivity and 83.3-100% specificity. Pasteurellaceae was primarily associated with the canonical variable for disease status QDA, followed by Staphylococcus (Table 4). Removal of any one of the eight primer sets resulted in significantly reduced QDA model performance on the validation sets.

Linear discriminant analysis (LDA) was used to separate samples by geographic location using 38 samples. Only one sample from Wyoming was misclassified by the LDA (2.6%, Table 4). Mycoplasma was primarily associated with the canonical variable for geographic location LDA, followed by Staphylococcus and Prevotellaceae. Only removal of the Mycoplasma primer set resulted in significantly reduced LDA model performance.

### Discussion

The results presented herein provide evidence of BBOSM alterations in the context of OSCC. These results support existing evidence that tissue microbiomes are altered in the context of neoplasia (Mitra, A, et al. Sci Rep 2015, 5:16865; Curty, G, et al. Int J Mol Sci 2019, 21; Turner, N. D, et al. Curr Gastroenterol Rep 2013, 15:346; van Vorstenbosch, R, et al. Metabolites 2022, 13; Wang, Z, et al. Front Microbiol 2020, 11:997; Hashimoto, K, et al. J Oral Microbiol 2022, 14:2105574). Ocular disease in cattle is infrequently studied despite a population of 98.8 million cattle head in the United States (Cattle; National Agricultural Statistics Service: Jul. 22, 2022). Bovine ocular disease causes both significant morbidity and economic loss for producers (Tsujita, H, et al. Vet Clin North Am Food Anim Pract 2010, 26:511-529). Diagnosing and treating cattle with OSCC is challenging as many of these animals graze on open pasture throughout the year with limited daily observation. Benefits of earlier diagnosis are likely to include improved treatment outcomes and reduced economic loss.

In this study, 16S rRNA gene sequencing and relative abundance analysis identified a single phylum (Euryarchaeota) at a lower abundance in OSCC-affected eyes. Euryarchaeota is a methanogenic archaea found within the gut of over 175 animal species (Thomas, C. M, et al. Nat Commun 2022, 13:3358) and humans (Horz, H. P, et al. Archaea 2010, 2010:967271). Previously thought to be pathogenic, these microorganisms are now thought to play a role in homeostasis (Horz, H. P, et al. Archaea 2010, 2010:967271), though their function in livestock species remains mostly unknown (Peng, Y, et al. Front Vet Sci 2022, 9:973508). Further studies are necessary explore the relationship between Euryarchaeota and bovine OSCC. While geographic differences in the relative abundance of Kiritimatiellaeota, Proteobacteria, and Euryarchaeota were also identified, these findings are extremely likely to be primarily driven by uneven sample size.

A recent study described significant bacterial abundance differences in the oral microbiome of saliva of patients with and without oral squamous cell carcinoma (Hashimoto, K, et al. J Oral Microbiol 2022, 14:2105574). The study noted a marked decrease in the abundance of Firmicutes and a marked increase in the abundance of Fusobacteria and Bacteroidetes in the presence of oral squamous cell carcinoma (Hashimoto, K, et al. J Oral Microbiol 2022, 14:2105574). A non-significant trend for decreased Firmicutes in OSCC samples was detected, consistent with this previous study. In addition, though not significant, there was a trend for elevations in Fusobacteria and Bacteroidetes for OSCC samples compared to normal samples, similarly to the cited study.

While useful as an exploratory technique, 16S rRNA gene sequencing analysis was found to be mostly unsuitable for categorization of samples by disease status in our study population. For example, three separate relative abundance beta diversity indices demonstrated no difference between normal eyes and OSCC-affected eyes. While 16S rRNA gene sequencing analysis has been widely utilized for investigation of altered microbiome composition in high biomass environments such as the gut, these techniques are less widely used in low biomass environments such as the ocular surface (Scott, E. M, et al. Vet Ophthalmol 2021, 24:4-11). Recognizing this, methods were deliberately utilized to ensure that the potentially significant impact of contamination was accounted for and prevented, where possible. Negative controls were acquired from the sampling environments and DNA extraction kit. Positive and negative controls were utilized during 16S rRNA gene sequencing with subsequent subtractive analysis performed. All laboratory-based sampled handling was performed in a laminar flow hood to prevent contamination. Despite these precautions, some degree of sample contamination is inevitable.

Recent methods of bacterial microbiome quantification using RT-PCR to create a gastrointestinal dysbiosis index (DI) have been described and validated (Sung, C. H, et al. J Feline Med Surg 2022, 24:e1-e12; AlShawaqfeh, M. K, et al. FEMS Microbiol Ecol 2017, 93; Suchodolski, J. S. Vet Clin Pathol 2022, 50 Suppl 1:6-17; Chaitman, J, et al. Front Vet Sci 2020, 7:192; Pilla, R, et al. J Vet Intern Med 2020, 34:1853-1866). This approach can provide clinically useful insights by accounting for unavoidable variation in the total amount of bacterial DNA recovered from the target tissue or material and providing quantification of bacterial genera, families, and/or species of interest (Chaitman, J, et al. Front Vet Sci 2020, 7:192; Pilla, R, et al. J Vet Intern Med 2020, 34:1853-1866). In addition, specific family and species resolution can be achieved using RT-PCR to identify and quantify bacterial targets. Indeed, these results demonstrate the utility of such an approach as the QDA model was able to readily categorize samples based on disease status. The combination of bacterial targets appears to be critical to the success of the QDA model, as removal of individual components markedly reduced sensitivity and specificity.

### Conclusions

The results presented herein provide evidence of BBOSM alterations in the context of OSCC. Relative abundance-based analysis identified 11 core BBOSM phyla. One of these phyla (Euryarchaeota) was found in significantly lower abundance in OSCC-affected eyes. RT-PCR and subsequent QDA allowed for categorization of disease status with a high degree of sensitivity and specificity.

### Example 2: A Diagnostic Test for Infectious Bovine Keratoconjunctivitis (IBK) in Cattle

### Technical Aspects of the Technology

### Samples Used for Training Database Creation

Samples were acquired using funding from the USDA AFRI. Conjunctival swab samples were acquired from 607 eyes of cattle housed at various locations in the USA; 227 with clinical signs consistent with IBK and 380 determined to be normal. Dry sterile swabs were used for sample collection; simple foam or flocked swabs are appropriate for this purpose. The swabs were immediately placed into a dry sterile tube and kept chilled for transportation. The inferior conjunctival fornix of each eye was sampled by vigorously rubbing the swab in this area for 2-5 seconds. Examinations were performed by a team of expert observers. Bacterial DNA was extracted from the swabs using a commercially available kit (in this case, the DNeasy PowerSoil Pro Kit from Qiagen was used; however, any such kit would also be appropriate) and quantified (in this case using a Nanodrop device; however, any such system of quantification would be appropriate). All samples were diluted with sterile water as necessary to achieve similar end concentrations (between 0.3 and 35 ng/μL). The extracted, diluted DNA was then utilized for qPCR.

### Primers and Processes Used for Diagnostic Panel

A custom set of primers and controls was utilized for qPCR using a real-time PCR system (in this case the QuantStudio™ 12K Flex Real-Time PCR System, 384-well block, desktop from Applied Biosystems; however, any such system would also be appropriate). Primer sets utilized for the novel panel can be found in Table 1.

The plate set up for qPCR was as follows: 1) Each plate includes triplicates for each clinical sample tested. Mean values for each CT value (the number of amplification cycles required for the fluorescent signal to exceed the basal threshold level) are then calculated from the triplicates where possible. If ‘undetermined’ values are obtained for any well containing a sample (not control), these are treated as a value of ‘60’ for the purposes of further analysis; 2) Each plate includes triplicate controls containing nuclease-free water and each of the primers listed above to ensure that contamination is not present in the primer mixes; 3) Each plate includes a known standard of E. coli (Microbial DNA standard from Escherichia coli (Sigma Aldrich, 10 ng/μL) or similar), for both E. coli primers (ref 8 above, in triplicate wells,) and universal bacteria primers (in triplicate wells). All other CT values on each plate are normalized to these values; 4) Each well on the plate (10 μL total) contains the following: SYBR green master mix 5 μL (PerfeCTa® SYBR® Green FastMix®, ROX™ Quantabio or similar), Forward primer 0.3 μL, Reverse primer 0.3 μL, Nuclease free water 0.9 μL, Sample DNA or E. coli standard 2.5 μL; 5) DNA from each clinical sample is separately plated (in triplicate) with primers for Bovine GAPDH, Moraxella bovis, Moraxella bovoculi, Mycoplasma, Staphylococcus, Pasteurellaceae, Prevotellaceae, Weeksellaceae and Universal Bacteria. The known E. coli standard is plated with both Universal Bacterial primers and Escherichia coli primers, to allow for quality assessment and comparison between qPCR runs; 6) Once each plate has been set up, covered and routinely centrifuged, the qPCR system is set to perform 40 cycles as follows: Hold stage (50° C. for 2 minutes, 95° C. for 10 minutes), PCR stage (95° C. for 15 seconds, 60° C. for 1 minute), Melt curve stage (continuous) (95° C. for 15 seconds, 60° C. for 15 seconds, 60 to 95° C. in a gradient of 0.05° C./s, 95° C. for 15 seconds); and 7) The results from each well are reported as CT values, which are averaged across the triplicates for each set of primers.

### Computational Model for Classification of Disease State

A computational model for classification of disease state was created as follows. The averaged CT values of eight primer sets were each subtracted from the averaged universal bacterial primer CT values to create eight normalized values (one for each set of primers). Following normalization, the data was analyzed by constructing a multitude of decision trees using the ‘random forest’ algorithm, comparing samples from normal eyes and samples from IBK eyes. The number of ‘trees per forest’ was set to 100 with the early stopping option allowed. The number of predictors sampled at each split was 6 with minimum and maximum splits per tree set at 10 and 200. Approximately 80% of the data (sample size=482; 185 IBK and 297 normal) were used for building the training model, which was initially found to have 89% sensitivity and 95% specificity.

### Validation Assessment of the Technology as a Diagnostic Tool

Following creation of the novel computational model for classification of disease state, data from samples identified as either IBK or normal were used to validate the model. The validation set from 20% of the samples (sample size=125; 42 IBK and 83 normal) was fit into the created algorithm with 69 trees constructed. The median number of splits was 67 with minimum and maximum splits per tree of 53 and 85. Using this validation set, the model was able to correctly predict if a sample was from an IBK eye or normal eye with 86% sensitivity and 90% specificity.

### Example 3: Alterations to the Bovine Bacterial Ocular Surface Microbiome in the Context of Infectious Bovine Keratoconjunctivitis

### Background

Infectious bovine keratoconjunctivitis (IBK), colloquially termed “pinkeye”, is the most common ophthalmic disease of cattle (Angelos J A. Vet Clin North Am Food Anim Pract 2015 31(1):61-79) and a major cause for morbidity in this species (Kneipp M. Vet Clin North Am Food Anim Pract 2021 37(2):237-252). First described in the late 1800s (Billings F S. Buffalo Med Surg J 1889 28(9):499-504; Penberthy J. J Comp Pathol and Ther 1897 10:263-264), this condition continues to be identified in the United States and globally (Dennis E J, et al. Vet Clin North Am Food Anim Pract 2021 37(2):355-369). IBK manifests clinically as blepharospasm, epiphora, corneal edema, vascularization, and corneal ulceration, and may progress to corneal perforation and subsequent vision loss (Kneipp M. Vet Clin North Am Food Anim Pract 2021 37(2):237-252). Pain and vision loss associated with this condition ultimately lead to decreased weight gain (Angelos J A. Vet Clin North Am Food Anim Pract 2015 31(1):61-79) and decreased milk production (Martins B C (2021) Infectious Bovine Keratoconjunctivitis. In: Gelatt K N (ed) Veterinary Ophthalmology, vol 2. John Wiley & Sons, Inc, pp 1998-2007). IBK is not only an animal welfare concern but also leads to economic losses for producers at slaughter (Brown M H, et al. J Vet Intern Med 1998 12(4):259-266; Funk L D, et al. J Am Vet Med Assoc 2014 244(1):100-106). Major economic losses, most recently reported at 150 million USD annually (Bartenslager A C, et al. Animal Microbiome 2021 3(1)), are incurred by producers as a result of treatment costs and loss of value due to decreased weight gain, decreased milk production, and corneal scarring (Kneipp M. Vet Clin North Am Food Anim Pract 2021 37(2):237-252).

Though more recently suggested to be an umbrella-term for a range of seemingly indistinguishable ocular diseases in cattle (Kneipp M. Vet Clin North Am Food Anim Pract 2021 37(2):237-252; Dennis E J, et al. Vet Clin North Am Food Anim Pract 2021 37(2):355-369), IBK is historically thought to be caused by Moraxella bovis, a gram-negative coccobacillus bacteria (Loy J D, et al. Vet Clin North Am Food Anim Pract 2021 37(2):279-293). This organism is the only organism to consistently produce IBK-like lesions in various experimental models when combined with corneal scarification (Aikman J G, et al. Vet Rec 1985 117(10):234-239; Henson J B, et al. Am J Vet Res 1960 21:761-766; Gould S, et al. Vet Microbiol 2013 164(1-2):108-115). IBK infection has also been associated with the presence of Moraxella bovoculi (Angelos J A, et al. Int J Syst Evol Microbiol 2007 57(Pt 4):789-795), Mycoplasma spp. (Schnee C, et al. Vet J 2015 203(1):92-96), and other pathogenic and opportunistic pathogens, though a definitive causal relationship has not been established (Loy J D, et al. Vet Clin North Am Food Anim Pract 2021 37(2):295-308). Treatment therefore often includes parenteral broad-spectrum antibiotics labeled for use in cattle with IBK (Angelos J A. Vet Clin North Am Food Anim Pract 2015 31(1):61-79). Vaccinations specific to these organisms as preventative measures have been produced and administered with little to no experimentally proven efficacy (Burns M J, et al. Vaccine 2008 26(2):144-152; Hille M M, et al. Vaccines (Basel) 2022 10(6)).

The bacterial ocular surface microbiome (OSM), though low in biomass, has been studied both in the eyes of normal animals (Darden J E, et al. PLoS One 2019 14(10):e0223859; Rogers C M, et al. PLoS One 2020 15(6):e0234313; Leis M L, et al. PLoS One 2021 16(2):e0247392; Santibanez R, et al. Animals (Basel) 2022 12(8); Scott E M, et al. PLoS One 2019 14(4):e0214877; Seyer L D, et al. Vet Ophthalmol 2022 25(4):297-306) and in the context of disease in humans (Zilliox M J, et al. Ocul Surf 2020 18(4):706-712; Gomes J A P, et al. Asia Pac J Ophthalmol (Phila) 2020 9(6):505-511; Aragona P, et al. Surv Ophthalmol 2021 66(6):907-925). Recognizing differences in the composition of the OSM in normal and diseased states allows identification of unique targets which may be exploited for treatment and prevention of disease.

Various methods of assessing the OSM are available including bacterial culture, 16S ribosomal ribonucleic acid (rRNA) gene sequencing, and real-time polymerase chain reaction (RT-PCR). Bacterial culture using ocular surface samples has been shown to have relatively low diagnostic utility, particularly when compared to results achieved by molecular diagnostic tools (Taravati P, et al. Curr Ophthalmol Rep 2013 1(4)). For example, suspected false-negative bacterial cultures have been identified in IBK cases, and therefore alternative or adjunctive testing is recommended (Loy J D, et al. Vet Clin North Am Food Anim Pract 2021 37(2):279-293). 16S rRNA gene sequencing utilizes conserved and hypervariable regions of bacterial genetic material to detect and classify a broad range of bacteria into amplicon sequence variants (ASVs), which are then used to evaluate relative taxonomic diversity richness and evenness of distribution through alpha- and beta-diversity indices (Chiarello M, et al. PLOS ONE 2022 17(2):e0264443; Janda J M, et al. J Clin Microbiol 2007 45(9):2761-2764; Mignard S, et al. J Microbiol Methods 2006 67(3):574-581; Banks K C, et al. Vet Ophthalmol 2019 22(5):716-725). However, assessing relative composition by use of sequencing alone may lead to incomplete characterization of the impact of mutual dependence and thus interaction of taxa (Vandeputte D, et al. Nature 2017 551(7681):507-511; AlShawaqfeh M K, et al. FEMS Microbiol Ecol 2017 93(11)). In addition, 16S rRNA gene sequencing is associated with higher costs and requires performance of advanced bioinformatic techniques (AlShawaqfeh M K, et al. FEMS Microbiol Ecol 2017 93(11)). Real-time polymerase chain reaction (RT-PCR) for semi-quantitative assessment of specific elements of bacterial ocular microbiome through amplification of primer-specific signals (Tsuji H, et al. Front Microbiol 2018 9:1417; Banks K C, et al. Vet Ophthalmol 2019 22(5):716-725), on the other hand, is recognized for its relative simplicity, accessibility, and cost-effectiveness (Jian C, et al. PLoS One 2020 15(1):e0227285). Clinically, quantitative PCR has been employed to create a mathematical model and subsequently a single numerical value, known as the dysbiosis index, to provide actionable information regarding changes to a patient's microbiome (AlShawaqfeh M K, et al. FEMS Microbiol Ecol 2017 93(11); Pilla R, et al. J Vet Intern Med 2020 34(5):1853-1866; Sung C H, et al. J Feline Med Surg 2022 24(6):e1-e12). However, drawbacks to RT-PCR when compared to 16S rRNA gene sequencing include a limited scope due to the selection of specific bacterial primers for assessment, as well as increased labor and time (Dreier M, et al. BMC Microbiol 2022 22(1):48). Yet relative abundance through sequencing and quantitative assessment through PCR may be considered complementary when studying the microbiome; sequencing provides a broad overview and suggests possible PCR primer targets, while quantitative PCR provides repeatable, quantitative data and allows identification of biases in analysis (Dreier M, et al. BMC Microbiol 2022 22(1):48). Combining the techniques of bacterial culture, 16S rRNA gene sequencing, and semi-quantitative RT-PCR when evaluating a specific microbiome may provide both a thorough overview and in-depth picture, allowing for a deeper understanding of the community under study. More specifically, wholly understanding microbiome dynamics may yield identification of the most appropriate target organism for treatments in the context of dysbiosis (Jian C, et al. PLoS One 2020 15(1):e0227285).

Reports investigating the bovine bacterial OSM through bacterial culture, 16S rRNA sequencing or PCR in the context of IBK exist. While historically Moraxella bovis was isolated most commonly in cases of IBK (Brown M H, et al. J Vet Intern Med 1998 12(4):259-266), one retrospective study found that a majority of IBK samples (600/1042) yielded viable Moraxella bovoculi (Loy J D, et al. J Vet Diagn Invest 2014 26(6):761-768). Another study of calves naturally infected with IBKfound through PCR testing that Moraxella bovoculi was more commonly detected than Moraxella bovis (O'Connor A M, et al. Vet Microbiol 2012 155(2-4):374-380). A separate study utilized 16S rRNA gene sequencing of calves naturally infected with IBK and reported minimal detectable differences in microorganism abundance between affected and control animals (Cullen J N, et al. Vet Microbiol 2017 207:267-279). Finally, a longitudinal study of 16S rRNA gene sequencing of calves naturally infected with IBK identified increased relative abundance of Mycoplasma and decreased relative abundance of Moraxella in the context of IBK (Bartenslager A C, et al. Animal Microbiome 2021 3(1)). To the authors' knowledge, a study combining the three techniques of culture, 16S rRNA gene sequencing, and RT-PCR has yet to be performed and may more completely elucidate microbiome dynamics in the context of IBK.

The purpose of this study was to comprehensively characterize the bovine bacterial OSM in the naturally-occurring IBK disease state as compared to Normal through a combination of bacterial culture, 16S rRNA gene sequencing, and RT-PCR. We hypothesize that each of the three utilized methods will detect significant differences in the bovine bacterial OSM based on disease status (IBK or Normal).

### Results

### Demographic Data

Six hundred and four individual eyes (Table 1) from cattle from 8 states (Table 2) and 17 farms (Table 3) were included in the study. Two hundred and twenty-eight eyes were diagnosed as having IBK disease, while 376/604 eyes were diagnosed as Normal. Chi-squared test was used to assess for significance of demographic data. There was no correlation identified between eye sampled (OS or OD) and disease status (Normal or IBK) (p=0.1652). A higher percentage of IBK eyes compared to Normal eyes were sampled from male cattle (54/109, 49.54% IBK) than were sampled from female cattle (174/495 eyes, 35.15% IBK) (p=0.0055). Age (three categories: <1 year old, 1 to 5 years old, and >6 years old) was independent of disease status (Normal or IBK) (p=0.1885). Most eye sampled (573/604) originated from animals aged<1 year old, which reflects the nature of meat and dairy production in the USA. Breed, when classified into one of two categories (those raised for beef or dairy purposes), was independent of disease status (p=0.9353).

### Bacterial Culture

In total, 387 culture swabs were obtained from 228 IBK eyes and 159 Normal eyes for in-vitro culture (Table 4). Associations were investigated by Chi-squared test. There were no significant differences in the frequency of positive Moraxella bovis isolation rates between groups (IBK or Normal) (p=0.1481). There were significant differences in the frequency of positive Moraxella bovoculi isolation rates between groups (IBK or Normal) (p<0.0001). There were no significant differences in the frequency of positive Moraxella osloensis, Proteus spp., Trueperella pyogenes, unspecified fungus, Bacillus spp., and mixed bacterial growth cultures between groups (IBK or Normal) (p>0.05).

16S rRNA Gene Sequencing: Bacterial Population Composition

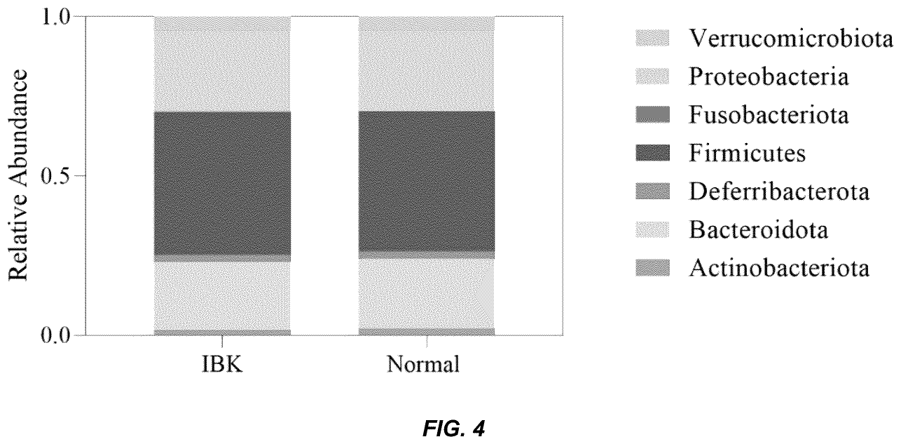

From 604 total conjunctival samples, 21,879 ASVs were determined using DADA2. Decontam was used to remove potential contaminant ASVs and ASVs encompassing less than 0.001% relative abundance were removed, leaving 20,191 ASVs for downstream analysis. The average number of high-quality reads per sample was 22,416. The seven most commonly identified phyla from all samples, encompassing 120 ASVs, were Actinobacteriota, Bacteroidota, Deferribacterota, Firmicutes, Fusobacteriota, Proteobacteria and Verrucomicrobiota (FIG. 4). Student's t tests or ANOVA tests were used to evaluate relative abundance.

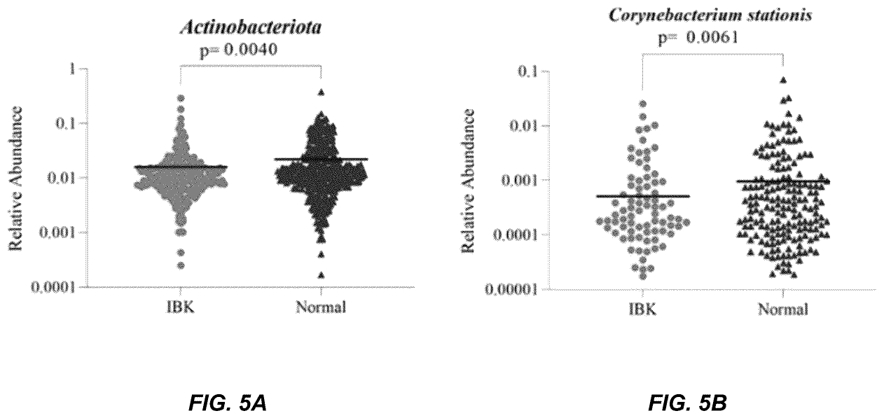

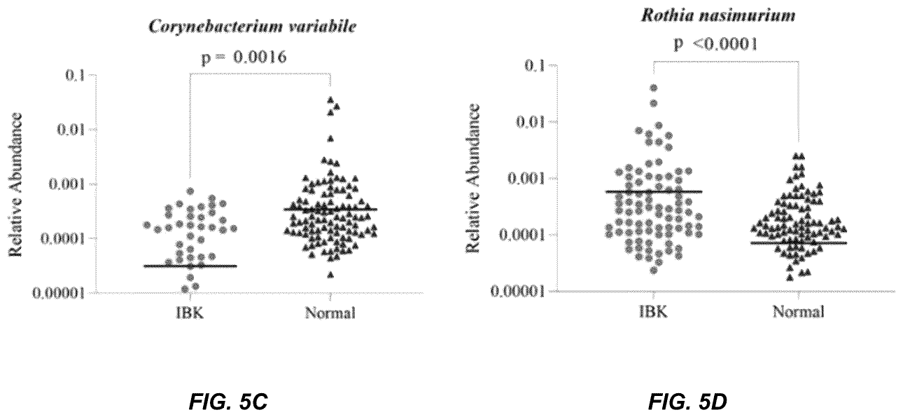

The overall relative abundance of Actinobacteriota was significantly greater (p=0.0045) in Normal eyes (mean±SD: 2.25%±3.09%) than IBK eyes (mean±SD: 1.61%±2.61%) (FIG. 5). Species within the Actinobacteriota phylum found at significantly higher relative abundance in Normal eyes than in IBK eyes included Corynebacterium stationis (mean±SD: Normal=0.098%±0.46%, IBK=0.051%±0.22%; p=0.0025) and Corynebacterium variabile (mean±SD: Normal=0.034%±0.25%, IBK=0.0031%±0.099%; p=0.0008) (FIG. 5). Rothia nasimurium, also within the Actinobacteriota phylum, was found at significantly higher relative abundance in IBK eyes (mean±SD: 0.058%±0.31%) than in Normal eyes (mean±SD: 0.0074%±0.025%) (p<0.0001) (FIG. 5).

The relative abundance of the Moraxella genus (of phylum Proteobacteria) was significantly higher in IBK eyes (mean±SD: 9.15%±13.65%) compared with Normal eyes (mean±SD: 2.62%±6.59%) (p<0.0001) (FIG. 6). The relative abundance of the Pasteurellaceae (of phylum Pseudomonadota) was significantly higher in IBK eyes (mean±SD: 13.83%±1.38%) compared with Normal eyes (mean±SD: 0.75%±0.07%) (p=0.0163).

Significant differences in relative abundance based on geographic location, sex, and breed (by purpose and individual breed) were noted. Relative abundance by geographic location (state and farm, evaluated separately) was significantly different for 7/7 of the most commonly identified phyla (p<0.0001). Individual breed (6 levels) was also a significant factor for 7/7 of the most commonly identified phyla (Verrucomicrobiota: p=0.0007; all other phyla: p<0.0001). Relative abundance by breed purpose (dairy or beef) was found to be significantly different for 7/7 phyla: relative abundance of Firmicutes and Actinobacteriota were increased in dairy cattle compared to beef cattle (p<0.0001); relative abundance of Bacteriodota, Deferribacterota, and Fusobacteriota were increased in beef cattle (p<0.0001); relative abundance of Proteobacteria (p=0.0012) and Verrucomicrobiota (p=0.0004) were increased in beef cattle. Relative abundance by sex was noted to be significantly different for 4/7 phyla: Actinobacteriota (p=0.0007), Firmicutes (p<0.0001), and Verrucomicrobiota (p=0.0175) were of higher relative abundance in female cattle (mean±SD: 2.19%±3.08%, 46.0%±20.21%, and 4.67%±2.57%, respectively) than male cattle (mean±SD: 1.15%±1.89%, 36.4%±17.33%, and 4.03%±2.49%, respectively), while Proteobacteria (p<0.0001) were of higher relative abundance in male cattle (mean±SD: 35.40%±22.45%) than female cattle (mean±SEM: 22.92%±19.10%).

16S rRNA gene sequencing: alpha-diversity

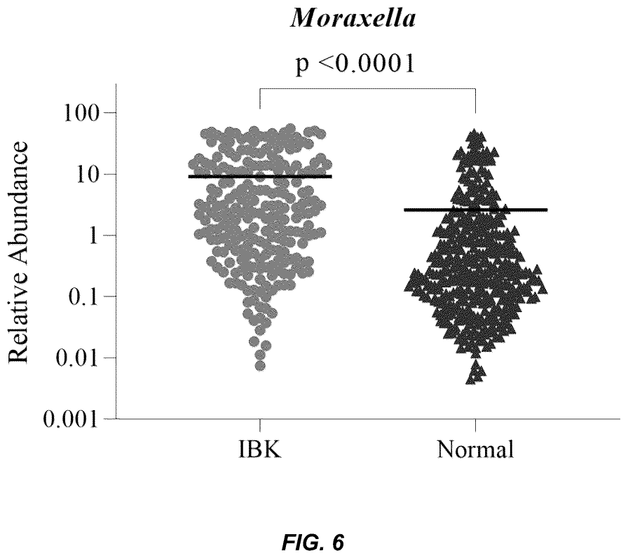

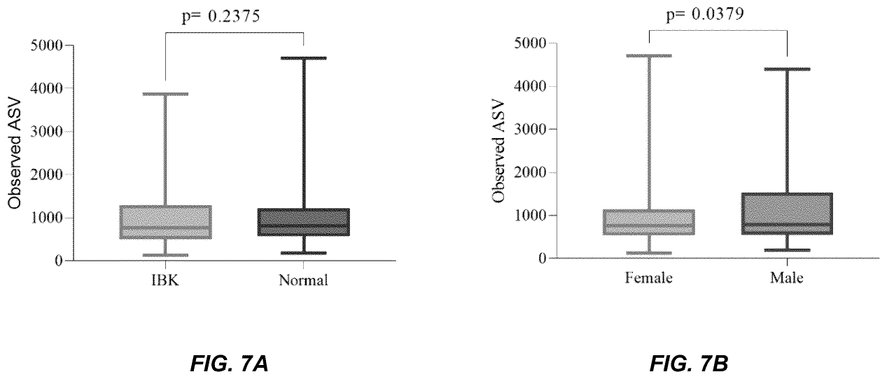

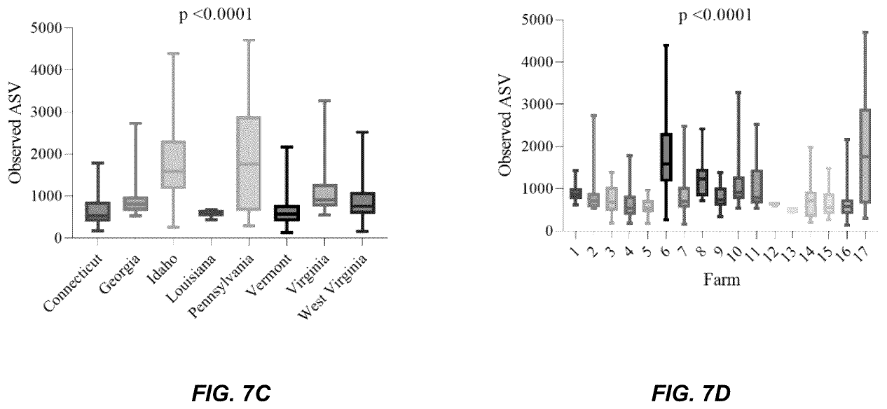

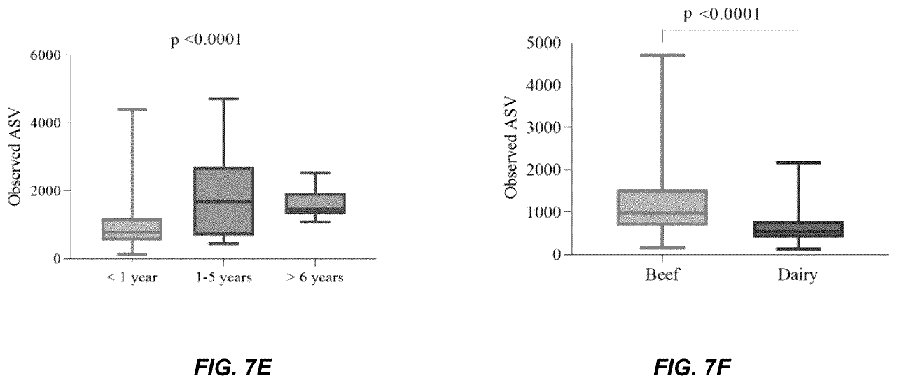

Non-parametric Kruskal-Wallis and Mann-Whitney U tests were utilized for the investigation of alpha-diversity (FIG. 7). There were no significant differences in alpha-diversity with regard to disease status (IBK or Normal) via any of the investigated indices, including Observed ASV, Chao1, Shannon, Simpson, and Faith's PD (p>0.05). There were significant differences in alpha-diversity with regard to geographic location (state and farm) and age by all investigated indices (p<0.0001) and by sex for Observed ASV (p=0.0379), Chao1 (p=0.0456), Pielou (p=0.0207), and Faith's PD (p=0.0187). There were significant differences in alpha-diversity with regard to breed (dairy and beef) for Observed ASV (p<0.0001), Chao1 (p<0.0001), Shannon (p=0.0003), Simpson (p=0.0181), and Faith's PD (p<0.0001).

16S rRNA Gene Sequencing: Beta-Diversity

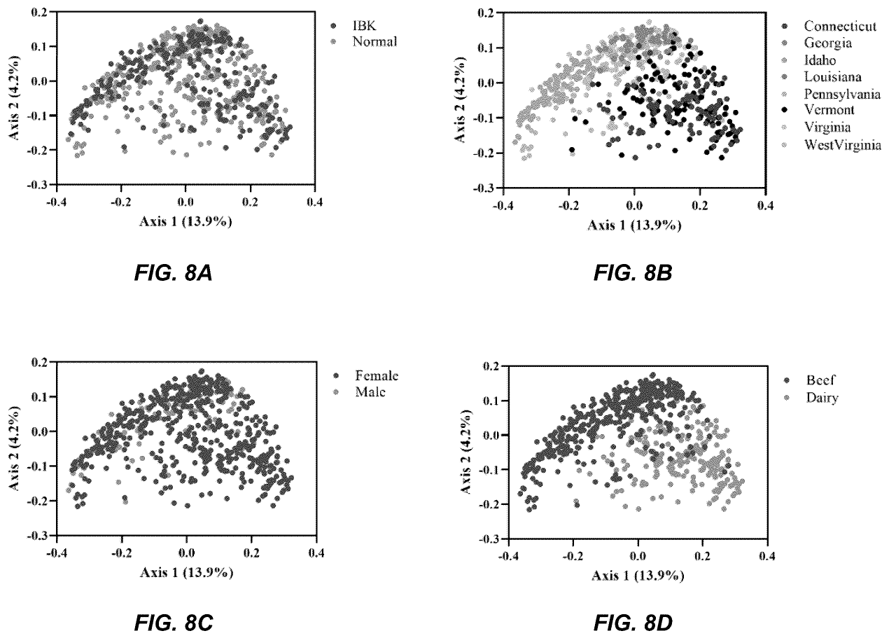

There were significant differences in bacterial beta-diversity identified by PERMANOVA by disease status (IBK or Normal) (unweighted unifrac: p=0.002; weighted unifrac analysis: p<0.001; Bray-Curtis: p<0.001; FIG. 8). However, significant differences in beta-diversity were also identified with regard to geographic location (state; unweighted unifrac, weighted unifrac, Bray-Curtis: p<0.001; FIG. 8), sex (unweighted unifrac, weighted unifrac, Bray-Curtis: p<0.001; FIG. 8), age (unweighted unifrac, weighted unifrac, Bray-Curtis: p<0.001; FIG. 8), and breed (beef and dairy: unweighted unifrac, weighted unifrac, Bray-Curtis: p<0.001; FIG. 8). When samples were selectively analyzed in smaller subgroups where the only known variable was disease status, beta diversity indices indicated significantly different microbiome composition in the majority of cases.

### RT-PCR

Two hundred and twenty-seven IBK and 369 Normal samples were evaluated by RT-PCR. Mann-Whitney U tests were used to evaluate the log DNA concentration against disease status. The average log concentrations of Moraxella bovis (IBK/Normal: 5.09/3.73; p<0.0001), Moraxella bovoculi (IBK/Normal: 5.14/3.79; p<0.0001), Pasteurellacaea (IBK/Normal: 5.37/4.76; p<0.0001), and Weeksellaceae (IBK/Normal: 6.38/6.14; p=0.0001) detected were significantly higher in IBK than in Normal eyes (Table 5). The average log concentration of Staphylococcus spp. was higher in Normal eyes than in IBK eyes (IBK/Normal: 3.83/4.60; p<0.0001). No significant difference in log concentrations by disease status (IBK or Normal) was observed for the remaining primer targets evaluated (Bov GAPDH, Mycoplasma, Prevotellaceae, universal bacteria) by RT-PCR (p>0.05) (Table 5).

### Classification Analysis Via Random Forest Algorithm

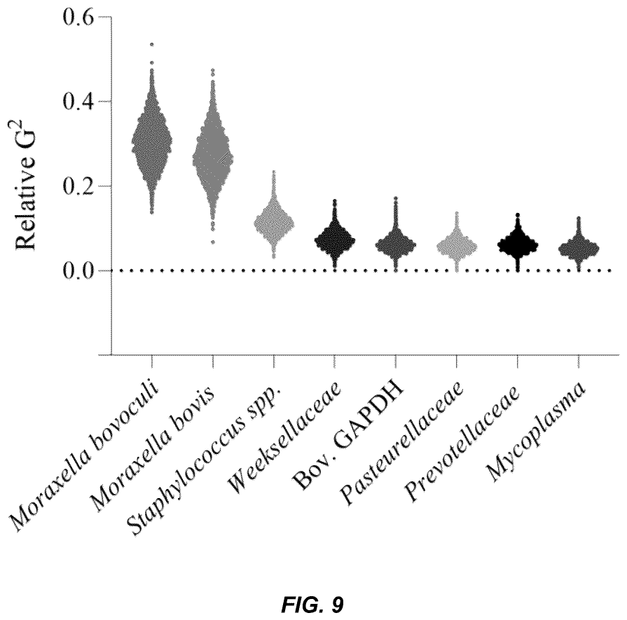

Data modeling was performed to categorize RT-PCR values by disease status (IBK or Normal) using training sets, with the resultant model then tested using validation sets. Validation set sensitivities ranged from 69.2-95.2%, and specificities ranged from 80.0-96.4%. The interpretation of primer contribution was calculated by relative deviance (G²) from the individual primer within each trial. The three most consistent primers contributing to the canonical variable to classify disease status were Moraxella bovoculi (22.1-42.5%), Moraxella bovis (21.2-42.6%), and Staphylococcus spp. (9.1-18.5%), while Pasteurellaceae (2.8-7.4%) Mycoplasma (2.1-6.5%) and Prevotellaceae (2.8-7.7%) were consistently the least three significant primers contributing to the canonical variable in the eight-primer set (FIG. 9).

### Discussion

In the present study, a large sample size (n=604 eyes from 17 farms in 8 US states) was acquired to comprehensively investigate the bovine bacterial OSM through bacterial culture, 16S rRNA gene sequencing, and RT-PCR to synergistically characterize the OSM in both normal cattle and cattle with IBK. In addition, RT-PCR value modeling was utilized to categorize samples as IBK or Normal. While historically Moraxella bovis has been considered the primary etiologic agent associated with IBK, vaccinations and antimicrobial treatments targeting this pathogen show limited efficacy in preventing and limiting disease (Burns M J, et al. Vaccine 2008 26(2):144-152; Hille M M, et al. Vaccines (Basel) 2022 10(6)). The present study confirms alterations in the relative abundance of Moraxella species between Normal eyes and IBK eyes but also suggests that numerous additional bovine bacterial OSM changes are present in IBK.

Aerobic bacterial culture was chosen as the first of three methods of OSM assessment due to frequent clinical use for confirmatory diagnosis of IBK (Brown M H, et al. J Vet Intern Med 1998 12(4):259-266). However, results of the present study indicate that bacterial culture alone is unlikely to provide meaningful data for clinical bovine bacterial OSM assessment. Both Moraxella bovis and Moraxella bovoculi are frequently isolated from both normal and IBK-affected eyes (Angelos J A. Vet Clin North Am Food Anim Pract 2015 31(1):61-79). In the present study, Moraxella bovis was isolated using samples from 7/228 (3.07%) of IBK eyes and Moraxella bovoculi was isolated using samples from 59/228 (25.88%) of IBK eyes; both organisms were isolated using samples from Normal eyes. As false negatives occur with simultaneous growth of multiple organisms in bacterial culture (Loy J D, et al. Vet Clin North Am Food Anim Pract 2021 37(2):279-293) (220/228 (96.49%) samples from IBK eyes yielded mixed bacterial growth in the present study), this technique has limited diagnostic potential in cattle with IBK. While it is recognized that bacterial culture is an extremely important established technique to identify the presence of viable bacteria, the results of this study indicate that, where possible, additional molecular diagnostic methods should be utilized for the diagnosis of IBK.

16S rRNA gene sequencing and relative abundance analysis was employed as the second of three methods of bovine bacterial OSM assessment in the context of IBK. Each of the most recently performed studies utilizing 16S rRNA sequencing to characterize the bovine bacterial OSM concluded that the bovine bacterial OSM is altered in the context of IBK (Bartenslager A C, et al. Animal Microbiome 2021 3(1); Cullen J N, et al. Vet Microbiol 2017 207:267-279; Anis E, et al. Vet Microbiol 2023 282:109752); results of the present study are consistent with this conclusion. The statistically significant proportional differences in bacterial relative abundance between Normal and IBK eyes frequently involved bacterial groups which composed a small proportion of the overall bacterial microbiome. However, it is considered likely that these bacterial groups represent ‘keystone taxa’ which drive community composition and function irrespective of their abundance (Amit G, et al. Nat Commun 2023 14(1):3951; Banerjee S, et al. Nat Rev Microbiol 2018 16(9):567-576). Through 16S rRNA gene sequencing, two species (Corynebacterium stationis and Corynebacterium variabile) from the phylum Actinobacteriota were noted to be present at significantly higher levels in Normal compared to IBK eyes, while a third species under the same phylum (Rothia nasimurium) was noted to be present at significantly higher levels in IBK eyes compared to Normal eyes. Corynebacterium species are part of the normal flora of the healthy human conjunctival sac and, while considered commensal, certain species have been shown to have pathogenic potential in immunocompromised patients (Aoki T, et al. Microorganisms 2021 9(2)). A study of the murine ocular microbiome reported the protective effect of a Corynebacterium species through elicitation of a protective immune response (Rigas Y, et al. Invest Ophthalmol Vis Sci 2023 64(2):19). In cattle, Corynebacterium stationis and Corynebacterium variabile have been isolated from the housing and milking environments of normal dairy cows, with Corynebacterium stationis most frequently isolated, mainly found in bedding and drinking troughs (Woudstra S, et al. Pathogens 2023 12(1)). Various Corynebacterium species have been suggested to have potentially protective probiotic effects in conditions such as nasal dysbiosis (Menberu M A, et al. Microbiol Res 2021 255:126927; Kiryukhina N V, et al. Probiotics Antimicrob Proteins 2013 5(4):233-238), vaginal dysbiosis (Gladysheva I V, et al. Microorganisms 2022 10(2); Gladysheva I V, et al. Probiotics Antimicrob Proteins 2023 15(3):588-600), and oral neoplasia (Shen X, et al. Bioengineered 2022 13(6):14094-14106). Corynebacterium spp. may play a protective role in IBK, and it is suggested that this possibility is explored in future studies. Rothia nasimurium is part of the normal flora of the human oropharynx and upper respiratory tract, though Rothia species have been implicated as an opportunistic pathogen in serious systemic illnesses in both immunocompromised and immunocompetent patients (Ramanan P, et al. J Clin Microbiol 2014 52(9):3184-3189; Fatahi-Bafghi M. Infect Genet Evol 2021 93:104877) and have been associated with bacterial endophthalmitis in humans (Alvarez-Ramos P, et al. Infect Dis Rep 2016 8(1):6320; Partner A M, Bhattacharya S, et al. Eye (Lond) 2006 20(4):502-503). Rothia nasimurium has also been shown to cause systemic disease in chickens (Zhang J, et al. Vet Sci 2022 9(12)) and ducks (Zhang J, et al. Vet Sci 2022 9(12); Wang M, et al. Infect Genet Evol 2021 90:104770). A study aiming to characterize the bovine ocular microbiota in the context of IBK in Mexico used 16S rRNA gene sequencing of cultured bacterial isolates and noted the presence of Corynebacterium species and Rothia nasimurium in some samples among other bacterial species, though the study did not specify if significant differences were detected between normal and IBK eyes (Rios-Alanis A M, et al. Rev Colomb Cienc Pecu 2021 34(1):18-28). Based on previous findings and the results presented herein, further research to determine the role of Rothia nasimurium in IBK is warranted.

The present study found relatively few statistically significant differences in bacterial relative abundance based on disease status (Normal or IBK) using 16S rRNA gene sequencing. Statistically significant differences in beta-diversity based on disease status were observed, with multiple variables (geographic location, sex, breed, and age) found to influence bovine bacterial OSM diversity. In common with a previous 16S rRNA gene sequencing ocular microbiome assessment (Deng Y, et al. Invest Ophthalmol Vis Sci 2020 61(2):47), geographic location appeared to significantly influence bovine bacterial OSM diversity in both Normal eyes and IBK eyes in the present study. A possible explanation for this finding is naturally occurring variation between subpopulations and the potential involvement of different IBK etiological agents in different locations. By including eyes from 8 different states and 17 different farms, the present study compiled a large enough sample size and a wide enough sample size to, consequently, describe the influence of geographic location on the bovine bacterial OSM. The context of geographic location must not be overlooked when characterizing the bovine bacterial OSM, and, therefore, all conclusions drawn from the present study must be considered with regard to the location in which samples were collected. In addition, the present study detected significant differences in overall relative abundance and alpha-diversity by sex and breed; therefore, these variables should also be considered when characterizing the bovine bacterial OSM.

A recent study by Anis et al utilizing 16S rRNA gene PCR and next generation sequencing analysis found differences in relative abundance of organisms in IBK eyes compared with control eyes (Anis E, et al. Vet Microbiol 2023 282:109752). For example, the relative abundance of Cardiobacteriaceae and Pasteurellaceae were noted to trend higher in eyes with IBK than in control eyes, and control eyes had elevated relative abundance of Sphingomonadaceae and Enterobacterioaceae compared to IBK eyes (Anis E, et al. Vet Microbiol 2023 282:109752). The relative abundance of three of these bacterial families, excluding Pasteurellaceae, were not observed to be significantly different between IBK and Normal samples in the present study when assessed using 16S rRNA gene sequencing. This may be due to differences in 16S rRNA gene sequencing protocols and analysis, variation of IBK between herds, or relative sample size. It should, however, be noted that the absolute and relative abundance of Pasteurellaceae was significantly elevated in IBK eyes compared to Normal eyes when evaluated with 16S rRNA gene sequencing and RT-PCR, respectively, in the present study. In common with the present study, Anis et al reported that 16S rRNA gene sequencing relative abundance assessment of IBK was limited by the inability to distinguish Moraxella bovis at the species level (Anis E, et al. Vet Microbiol 2023 282:109752). This further highlights the importance of utilizing multiple approaches to characterize the bovine bacterial OSM in the context of IBK.

RT-PCR was utilized as the third and final method of bovine bacterial OSM assessment in the present study. Copy numbers of DNA from Moraxella bovis, Moraxella bovoculi, Pasteurellaceae and Weeksellaceae were significantly higher in IBK eyes compared to Normal eyes. Staphylococcus spp. copy numbers were significantly higher in Normal eyes compared to IBK eyes. RT-PCR cycle threshold values were then utilized for classification analysis using a random forest algorithm to categorize eyes by disease status. In addition to possibly representing an improved confirmatory test for IBK, it is possible that this assay could be utilized in the future to identify environmental sources of IBK at individual farms. In the present study, validation set sensitivities and specificities ranged from 69.2-95.2%, and 80.0- 96.4%, respectively, indicating that samples analyzed with this primer set and mathematical model may predict disease status with moderate to high sensitivity and specificity. A ‘dysbiosis index’ to assess fecal microbiome alterations in veterinary patients, which utilizes a similar approach, has been employed in numerous studies (AlShawaqfeh M K, et al. FEMS Microbiol Ecol 2017 93(11); Pilla R, et al. J Vet Intern Med 2020 34(5):1853-1866; Sung C H, et al. J Feline Med Surg 2022 24(6):e1-e12; Chaitman J, et al. Front Vet Sci 2020 7:192). It is therefore postulated that the present study's use of classification analysis may be considered for the development of future techniques to study, diagnose, and make treatment recommendations for cattle with IBK.

There are several limitations of the present study, including sample collection techniques, inherent variations in 16S rRNA gene sequencing and analysis, bacterial culture techniques, validation of assays with no known standard and sample categorization. Samples were collected from cattle naturally affected with IBK while being handled for other management reasons at each farm. Out of necessity, this required rapid sample collection and thorough but brief ocular examinations by a trained veterinarian observer. Gloves were worn and changed between animals, but full, sterile personal protective equipment could not be utilized. We carefully considered this element of study design before performing the study and it was decided that the method of sample collection which was ultimately chosen represented the most realistic approach. As with any study utilizing 16S rRNA gene sequencing and analysis, variations in sample processing and analysis are likely to have influenced the results. This prevents direct comparison of our results to studies of a similar nature. For this reason, we collected a large sample size from a geographically diverse group of animals. To ensure optimal conditions for transportation of viable bacteria for subsequent in-vitro culture, we worked with experienced bacteriologists to design this element of the study. However, samples did require transportation (chilled) prior to initiation of processing, which could have led to lower numbers of viable bacteria being detected. The Normal samples utilized for bacterial culture originated from animals with contralateral IBK. As such, it is possible that the composition of the bacteria identified using culture was affected by the presence of contralateral disease, despite being carefully clinically examined and found to be Normal. As is outlined in the methods section, primers were used to identify various bacterial groups using RT-PCR. We adapted and partially validated (using dilutions of a pure culture of M. bovis, but not M. bovoculi) the use of primers from Zheng et al. (Zheng W, et al. J Microbiol Methods 2019 160:87-92) for identification of Moraxella bovis. Additional validation would be required to assess specificity of the Moraxella primers which were utilized in the present study. Despite concerted efforts, a suitable standard to validate the Weeksellaceae primers (identified using predictive local alignment) was not identified. As such, the values from this component of the RT-PCR panel cannot be considered to be specific for Weeksellaceae. Finally, IBK was diagnosed based on the presence of compatible clinical signs, rather than the presence of a single organism (eg, Moraxella bovis). This element of study design was deliberate, based on previously discussed evidence that Moraxella bovis is not present in many suspected cases of IBK (Loy J D, et al. J Vet Diagn Invest 2014 26(6):761-768; Loy J D, et al. Vet Clin North Am Food Anim Pract 2021 37(2):295-308; Angelos J A. Vet Clin North Am Food Anim Pract 2015 31(1):61-79; Angelos J A, et al. Int J Syst Evol Microbiol 2007 57(Pt 4):789-795).

Sampling bias also represents a possible limitation of the present study. Affected herds for sampling and inclusion were identified by specifically seeking animals with IBK. Therefore, all farms which were visited during the sample collection phase were known to have IBK affected cattle and as such, comparison statistics reported herein involving demographics and disease status are likely to be impacted by sampling bias. In addition, Normal eyes from IBK unaffected cattle (cattle with two Normal eyes) were sampled at each location, and the number of Normal cattle sampled at each location was not consistent due to animal availability. Sampling bias may therefore explain certain statistically significant findings, such as the higher number of IBK-affected eyes in male cattle compared to Normal eyes in male cattle. Sampling bias also likely contributed to the number and types of breeds included in the study: a large number of Angus cattle affected by IBK were included when compared to breeds such as Hereford cattle. Angus cattle have been studied for genetic predisposition to IBK development (Kizilkaya K, et al. BMC Proc 2011 5 Suppl 4 (Suppl 4):S22), though Hereford cattle are a breed considered to have high susceptibility and increased predisposition, theorized to be related to periocular pigmentation or heredity (Pugh G W, et al. J Vet Res 1986 50(2):259-264; Snowder G D, et al. J Anim Sci 2005 83(3):507-518). A large variation in sample sizes of male cattle and female cattle sample may be related both to sampling biases described above as well as to an unbalanced ratio of male and female cattle across the United States. Finally, a large variation in sample size with regard to age may be related both to sampling biases described above as well as to a previously documented predisposition for this disease to affect young calves (Dennis E J, et al. Vet Clin North Am Food Anim Pract 2021 37(2):355-369).

### Conclusions

This study provides further evidence that the bovine bacterial OSM is altered in the context of IBK, indicating the involvement of a variety of bacteria in addition to Moraxella bovis, including Moraxella bovoculi and R. nasimurium, among others. Actinobacteriota relative abundance is altered in IBK, providing opportunities for novel therapeutic interventions. While RT-PCR modeling provided limited further support for the involvement of Moraxella bovis in IBK, this was not overtly reflected in culture or RA results. RT-PCR modeling demonstrates potential as a cost-effective method to reliably confirm IBK.

### Methods

### Subject Selection and Examination

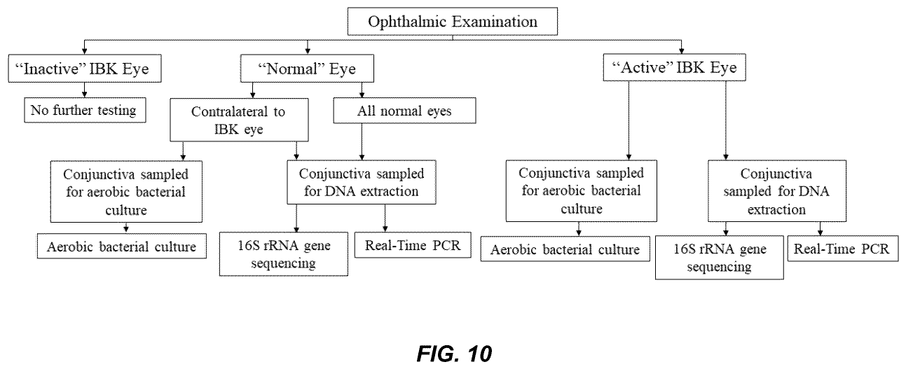

The study was approved by the Louisiana State University (LSU) Institutional Animal Care and Use Committee (Animal Use Protocol 19-092). A flow chart for visualization of the experimental process may be found in FIG. 10. Outbreaks of IBK were identified by telephone calls and emails to veterinary schools, veterinary practices, and producer organizations within the United States. Herd examinations and sample collection were performed between May and October 2021. Consent of the herd owner or equivalent representative was obtained prior to performing conjunctival sampling of the eyes of affected and normal cattle. All animals had received no treatment in the 7 days prior to examination and sample collection. Where possible, visits were made in conjunction with a visit by the local responsible veterinarian who treated the animals immediately following conjunctival sample collection. Calves and adult cattle of both dairy and beef purpose were included in the study, with breed and age recorded as reported by farm personnel. Age was divided into three categories of <1 year old, 1-5 years old, and over 6 years old. Cattle for dairy production were housed in open-air barns and were handled frequently, while cattle for beef production were on pasture and handled relatively infrequently.

### Sample Collection

Ophthalmic examination was performed by a trained veterinary observer. Adult cattle were held in a chute with a head catch for examination, while younger calves were physically restrained by farm staff. The eyes were categorically diagnosed as ‘Normal’ or ‘IBK’. Only eyes with evidence of active IBK were included in the IBK group. An eye with two or more of the following clinical signs in the context of a herd of two or more cattle with similar signs was considered to have active IBK disease: blepharospasm, epiphora or ocular discharge, ulcerative keratitis, chemosis, conjunctival hyperemia, corneal vascularization, corneal edema, corneal infiltrate, and corneal perforation with or without iridial prolapse. Eyes with evidence of corneal scarring without active inflammation (inactive IBK) were not included in the study population. Only eyes diagnosed as IBK or Normal were included, and therefore both eyes of some cattle were not included due to the presence of inactive IBK. Following sample collection, eyes were evaluated as individual entities and were not kept paired for analysis. Animals with clinical signs indicative of a possibly unrelated ocular disease process (e.g. exophthalmos) were excluded from the study population.

If at least one eye of an animal was determined to be Normal, the lower conjunctival fornix of the normal eye(s) was sampled vigorously with two DNA buccal swabs (Isohelix Swab Pack, MidSci, St Louis, MO) simultaneously. The swabs were placed in a 15 mL centrifuge tube (VWR, Radnor, PA) prior to being placed on ice for storage for later DNA extraction. If at least one eye of an animal was determined to be actively affected by IBK, the lower conjunctival fornix of the IBK eye was sampled vigorously with DNA buccal swabs (Isohelix Swab Pack, MidSCi, St Louis, MO) followed by a bacterial culture swab (Copan Diagnostic ESwab, Copan Diagnostics, Murrieta, CA) with each swab type then placed in its respective tube. If the contralateral eye of an IBK eye was also diagnosed as active IBK or Normal, both eyes were sampled for bacterial culture. If the contralateral eye was diagnosed as inactive IBK, only the active IBK eye was sampled for bacterial culture. If both eyes of an animal were diagnosed as Normal, neither eye was sampled for bacterial culture. The samples were then stored on ice for later DNA extraction and to be sent off for culture, respectively. Non-sterile gloves (VWR, Radnor, PA) were worn and changed between each animal during sample collection. An environmental control for use in relative abundance analysis was created at each sampling location by exposing two DNA buccal swabs to the air for approximately five seconds with immediate storage in a 15 mL tube on ice for later DNA extraction. All samples for DNA extraction were shipped to LSU School of Veterinary Medicine directly from the farm. Samples for bacterial culture were shipped directly to the Texas Veterinary Medical Diagnostic Laboratory (TVMDL) for processing.

### Bacterial Culture

Culture swabs were shipped on ice directly from sampling locations to the TVMDL for aerobic culture. For isolation of bacteria, conjunctival swab samples (n=387) were streaked onto two 5% sheep blood agar (Hardy Diagnostics, USA) plates and incubated aerobically with 10% CO₂ at 37° C. for 48 hours. All the culture plates were read at 24 hours and 48 hours for isolation of bacteria. Initial identification of different bacteria were based on colony morphologies on plates, and different biochemical test results including oxidase, catalase, indole, carbohydrate fermentation and gram staining. Gram stain and oxidase (BD Diagnostics, USA) tests were performed on all the Moraxella spp. suspected isolates producing greyish-white and hemolytic colonies. Moraxella spp. are gram negative cocci and oxidase positive. Finally, identification of different bacteria up to genus or species level were based on matrix-assisted laser desorption-ionization time of flight mass spectrometry (MALDI-TOF MS) (Bruker Daltonics, Germany) score results. A score of 2.3 to 3.0 was considered a highly probable species identification, and a score of 2.0 to 2.299 was considered a secure genus identification and probable species identification. Final identification of all bacterial isolates was based on the agreement between biochemical and MALDI-TOF MS results. When mixed bacterial colonies with no predominant colony types were present on bacterial culture plates, these were reported as ‘mixed bacterial growth’. For the samples when Moraxella spp. were not isolated, those were reported as “negative culture Moraxella spp.”

### DNA Extraction

DNA Extraction from each swab used for 16s rRNA gene sequencing and RT-PCR analysis was performed using the DNeasy PowerSoil Pro Kit (QIAGEN GmbH, Hilden, Germany) according to the manufacturer's instructions. DNA was extracted from conjunctival swabs, environmental control samples, and from extraction control samples created by replicating the protocol in the absence of swabs. A filtered laminar flow cabinet (The Clone Zone, USA/Scientific, Inc., Ocala, Florida, USA) was used to perform the extractions. Eluted DNA sample concentrations were calculated (NanoDrop One Microvolume UV-Vis Spectrophotometer, ThermoFisher Scientific, Waltham, MA), and samples were stored at −80° C.

16S rRNA Gene Sequencing and Analysis

Sequencing was performed by the LSU School of Medicine Microbial Genomics Resource Group. The AccuPrime Taq high fidelity DNA polymerase system (Invitrogen, Carlsbad, CA) (Table 6) was used to perform two steps of amplification for sequencing library preparation. Amplicon library preparation included processing negative controls (DNA extraction, environment, and PCR amplification) and a positive control (Microbial mock community HM-276D, BEI Resources, Manassas, VA). Twenty nanograms of genomic DNA and gene-specific primers with Illumina adaptors were used to amplify the hypervariable V4 region. PCR included steps listed in Table 7. AMPure XP beads with beads added as 0.85× the PCR volume were used to purify PCR products (targeting approximately 390 bp DNA). Using the same PCR conditions and primers with different molecular barcodes, 4 μL of purified amplicon DNA from the previous step was amplified for 8 cycles. AMPure XP (Beckman Coulter, Indianapolis, IN) beads were used to purify the indexed amplicon libraries, which were then quantified using Quant-iT PicoGreen (Invitrogen), normalized, and pooled. KAPA Library Quantification Kit (Kapa Biosystems, Cape Town, South Africa) was used to quantify the pooled library, followed by dilution and denaturation as per Illumina guidelines. As a quality control and to increase diversity of the 16S rRNA amplicon library, ten percent Illumina PhiX was added to the sequencing library. A 2×250 bp V2 sequencing kit was used to perform paired-end sequencing using Illumina MiSeq (Illumina, San Diego, CA). Quality analysis was performed through transfer of the sequencing reads to Illumina's BaseSpace. Further bioinformatics analysis was performed with the generated raw FASTQ files.

Sequencing reads from FASTQ files were imported into R version 4.2.0. Reads were then processed with DADA2 version 1.22.0. Read quality profiles were examined to select appropriate trimming and filtering parameters and were set to trim 20 bp (left) of each read and to truncate reads to 240 bp (both forward and reverse) to remove low quality tails. The standard DADA2 workflow, including error learning and sample inference for forward and reverse reads followed by merging of sequence variants, was utilized. The ‘removeBimeraDenovo’ process was used to remove chimeric sequence variants, and sequence variants outside of the expected amplicon size range of 249 to 256 bp were removed as well. The remaining sequence variants were placed into a sequence table with read counts ranging from 816 to 143,459. The SILVA database v138 was used to classify taxonomy, and a Phyloseq (McMurdie P J, et al. PLoS One 2013 8 (4):e61217) object was constructed using imported mapping information. Phyloseq (McMurdie P J, et al. PLoS One 2013 8 (4):e61217) version 1.38.0 was used to perform downstream analysis. Decontam (Davis N M, et al. Microbiome 2018 6(1):226) version 1.14.0 was used to identify and remove suspected contaminant ASV with the prevalence method (default parameters). Remaining ASVs with a mean relative abundance of less than 104 across all samples were filtered with an abundance filter.

### Real-Time PCR

RT-PCR for selected bacterial familes and species (Table 1) was performed using remaining eluted DNA and PerfeCTa SYBR Green FastMix, ROX (VWR, Radnor, PA) (Table 8). Bacterial target primer pairs were chosen based on previous clinical reports and exploratory 16S rRNA gene sequencing relative abundance analyses obtained from cattle with IBK (Cullen J N, et al. Vet Microbiol 2017 207:267-279; Bartenslager A C, et al. Animal Microbiome 2021 3(1); Zheng W, et al. J Microbiol Methods 2019 160:87-92). Eluted DNA concentrations from eyes of the same animal were standardized through dilution with molecular grade water (VWR, Radnor, PA) with an overall range of 0.3 to 34.3 ng/μl. DNA samples were individually combined with nine different primer pairs and analyzed in triplicate. The RT-PCR panel performed included the following primer (IDT, Coralville, IA) targets for each sample: bovine GAPDH (Menzies M, et al. Vet Immunol Immunopathol 2006 109(1-2):23-30), Moraxella bovis (Zheng W, et al. J Microbiol Methods 2019 160:87-92), Moraxella bovoculi (Zheng W, et al. J Microbiol Methods 2019 160:87-92), Mycoplasma (van Kuppeveld F J, et al. Appl Environ Microbiol 1992 58(8):2606-2615), Pasteurellaceae (Bootz F, et al. Lab Anim Sci 1998 48(5):542-546), Prevotellaceae (Stevenson D M, et al. Appl Microbiol Biotechnol 2007 75(1):165-174), Staphylococcus (Morot-Bizot S C, et al. J Appl Microbiol 2004 97(5):1087-1094), Weeksellaceae, and ‘universal bacteria’ (AlShawaqfeh M K, et al. FEMS Microbiol Ecol 2017 93(11)). The universal bacteria primer was used for data normalization, while the bovine GAPDH primer was utilized as a reference for host DNA in proportion to the total bacterial DNA extracted. Escherichia coli standard (10 ng/μL, Sigma-Aldrich, Saint Louis, MO) and molecular grade water (VWR, Radnor, PA) were used as positive and negative controls, respectively. To allow for standardization between runs and relative quantification of non-E. coli bacterial groups, the E. coli standard was plated in triplicate with both ‘universal bacteria’ primers and E. coli primers (Sidjabat H E, et al. J Antimicrob Chemother 2006 57(5):840-848). The RT-PCR run consisted of steps listed in Table 9. Using an E. coli standard on the same RT-PCR plate, the RT-PCR data was expressed as the log amount of DNA in atto-gram (10⁻¹⁸ g, ag) for each primer pair per 10 ng of isolated total DNA (Gafen H B, et al. Animals (Basel) 2023 13(12)). Within each sample, the C_{T} values of each primer set were normalized by universal bacteria C_{T} values for further analyses.

### Classification Analysis Via Random Forest Algorithm

The C_{T} values of eight primer sets (Bovine GAPDH included) were normalized by the universal bacteria primer C_{T} values generated for the same sample. The data was further analyzed using a random forest algorithm (Ho. Proceedings of 3rd International Conference on Document Analysis and Recognition 1995 1:278-282) to categorize Normal and IBK samples using commercial software (JMP Pro 16.2.0). The number of ‘trees per forest’ was set to 100 with the early stopping option allowed. The number of predictors sampled at each split was 6 with minimum and maximum splits per tree set at 10 and 200. Parameters (sensitivity and specificity from the validation sets and the relative deviance (G²) from the training set) were reported with 95% confidence limit generated via 2500 runs. Approximately 80% (n=459-498) and 20% (n=98-137) of the total data were used for building the training model, and validation set, respectively. Sensitivity and specificity values were calculated from each run for the training and validation sets separately.

### Statistical Analysis

Commercial software (JMP Pro 16.2.0 and R Statistical Software v4.1.3) was used to perform all statistical analyses. Chi-squared test was used to check associations between culture results, eye sampled (right or left), geographic location, and disease status. One way ANOVA and student's t tests were used to evaluate abundance against disease state, geographic location (state and farm), sex, age, and breed. Logarithmic transformation was performed for data that did not meet the normality criteria. Normality of residuals from the parametric models were accessed and confirmed by examining standardized residual and quantile plots. Data are presented as mean±SD. Kruskal-Wallis tests or Mann-Whitney tests against disease status, geographic location, breed, and sex were used to analyze alpha-diversity from 16S rRNA gene sequencing, and Log DNA concentration from RT-PCR. Beta-diversity indices (standard weighted unifrac analysis, unweighted unifrac analysis, and Bray-Curtis analysis) were evaluated via permutational multivariate analysis of variance (PERMANOVA) using vegan R package 2.6.2. Statistical significance was set at p<0.05

TABLE 1
Demographic data, including total samples collected, and variables recorded
such as eye, sex, age, breed purpose, and individual breeds.

| | Total | | | | | Statistical |
|---|---|---|---|---|---|---|
| | Number | Number of | | Number of | | Significance |
| Category | of Eyes | IBK Eyes | | Normal Eyes | | (Chi-Squared) |
| Total Number | 604 | 228 | (37.75%) | 376 | (62.25%) | |

of Eyes

Eye Sampled

| | | | | | | |
|---|---|---|---|---|---|---|
| Right eye | 296 | 120 | (40.54%) | 176 | (59.46%) | P = 0.1652 |
| Left eye | 308 | 108 | (35.06%) | 200 | (64.94%) | |

Sex

| | | | | | | |
|---|---|---|---|---|---|---|
| Female | 495 | 174 | (35.15%) | 321 | (64.85%) | P = 0.0055 |
| Male | 109 | 54 | (49.54%) | 55 | (50.46%) | |

Age

| | | | | | | |
|---|---|---|---|---|---|---|
| <1 year old | 573 | 221 | (38.57%) | 352 | (61.43%) | P = 0.1885 |
| 1-5 years old | 24 | 5 | (20.83%) | 19 | (79.17%) | |
| >6 years old | 7 | 2 | (28.57%) | 5 | (71.43%) | |

Purpose/Breed

| | | | | | | |
|---|---|---|---|---|---|---|
| Beef cattle | 388 | 146 | (37.63%) | 242 | (62.37%) | |
| Angus | 371 | 140 | (37.74%) | 231 | (62.26%) | P = 0.9353 |
| Hereford | 15 | 5 | (33.33%) | 10 | (66.66%) | |
| Charolais | 2 | 1 | (50%) | 1 | (50%) | |
| Dairy cattle | 216 | 82 | (37.96%) | 134 | (62.04%) | |
| Brown Swiss | 8 | 4 | (50%) | 4 | (50%) | P = 0.9353 |
| Holstein | 202 | 77 | (38.11%) | 125 | (61.88%) | |
| Jersey | 6 | 1 | (16.67%) | 5 | (83.33%) | |

Chi-squared test was used to assess for significance.

TABLE 2
Location data by US state, including total samples collected.

| | Total Number | Number of | Number of |
|---|---|---|---|
| Location (State) | of Eyes | IBK Eyes | Normal Eyes |
| Connecticut | 103 | 38 | 65 |
| Georgia | 47 | 8 | 39 |
| Idaho | 125 | 69 | 56 |
| Louisiana | 6 | 3 | 3 |
| Pennsylvania | 20 | 5 | 15 |
| Vermont | 113 | 44 | 69 |
| Virginia | 41 | 7 | 34 |
| West Virginia | 149 | 54 | 95 |

TABLE 3
Location data by farm, including total number of eyes sampled.

| | Total Number | Number of | Number of |
|---|---|---|---|
| Location (Farm) | of Eyes | IBK Eyes | Normal Eyes |
| Farm 1 | 26 | 3 | 23 |
| Farm 2 | 21 | 5 | 16 |
| Farm 3 | 27 | 3 | 24 |
| Farm 4 | 65 | 26 | 39 |
| Farm 5 | 24 | 5 | 19 |
| Farm 6 | 125 | 69 | 56 |
| Farm 7 | 78 | 42 | 36 |
| Farm 8 | 19 | 4 | 15 |
| Farm 9 | 14 | 3 | 11 |
| Farm 10 | 41 | 7 | 34 |
| Farm 11 | 11 | 2 | 9 |
| Farm 12 | 4 | 2 | 2 |
| Farm 13 | 2 | 1 | 1 |
| Farm 14 | 31 | 9 | 22 |
| Farm 15 | 38 | 12 | 26 |
| Farm 16 | 58 | 30 | 28 |
| Farm 17 | 20 | 5 | 15 |

TABLE 4
Aerobic bacterial culture results.

| | IBK Eyes: | | IBK Eyes: | | Normal Eyes: | | Normal Eyes: | | Statistical |
|---|---|---|---|---|---|---|---|---|---|
| | Positive | | Negative | | Positive | | Negative | | significance |
| Category | Culture | | Culture | | Culture | | Culture | | (Chi-squared) |
| Moraxella bovis | 7 | (3.07%) | 221 | (96.93%) | 1 | (0.63%) | 158 | (99.37%) | P = 0.1481 |
| Moraxella bovoculi | 59 | (25.88%) | 169 | (74.12%) | 7 | (4.40%) | 152 | (95.60%) | P < 0.0001 |
| Moraxella osloensis | 1 | (0.44%) | 227 | (99.56%) | 0 | (0%) | 159 | (100%) | P > 0.05 |
| Trueperella pyogenes | 4 | (1.75%) | 224 | (98.25%) | 0 | (0%) | 159 | (100%) | P > 0.05 |
| Unspecified fungus | 10 | (4.39%) | 218 | (95.61%) | 12 | (7.55%) | 147 | (92.45%) | P > 0.05 |
| Bacillus spp. | 7 | (3.07%) | 221 | (96.93%) | 2 | (1.26%) | 157 | (98.74%) | P > 0.05 |
| Proteus spp. | 1 | (044%) | 227 | (99.56%) | 0 | (0%) | 159 | (100%) | P > 0.05 |
| Mixed bacterial growth | 220 | (96.49%) | 8 | (4.51%) | 156 | (98.11%) | 3 | (1.89%) | P > 0.05 |

Chi-squared test was used to assess for significance.

TABLE 5
Relative DNA quantities obtained by PCR
and evaluated by Mann-Whitney U tests.

| | Median (Min-Max) Log DNA (ag = 10⁻¹⁸ g) | | | |
|---|---|---|---|---|
| | per 10 ng isolated total DNA | | | |
| | Disease status | | | |
| Target Primer | Normal (n = 369) | | IBK (n = 227) | |
| Bov. GAPDH | 7.18 | (4.99-8.41) | 7.23 | (4.85-7.83) |
| Moraxella bovis | 3.73 | (0.00-6.63)* | 5.09 | (0.00-8.34)* |
| Moraxella bovoculi | 3.79 | (0.00-7.10)* | 5.14 | (0.00-7.63)* |
| Mycoplasma | 7.20 | (3.72-8.85) | 7.21 | (3.74-9.16) |
| Staphylococcus | 4.60 | (0.00-9.19)* | 3.83 | (0.00-9.68)* |
| Pasteurellaceae | 4.76 | (0.00-7.85)* | 5.37 | (0.00-8.42)* |
| Prevotellaceae | 6.01 | (4.48-8.41) | 6.12 | (4.64-8.10) |
| Weeksellaceae | 6.14 | (0.00-9.00)* | 6.38 | (0.00-8.04)* |
| Universal bacteria | 8.19 | (6.63-10.30) | 8.23 | (6.85-10.30) |

Asterisk (*) indicates significant difference with respect to disease status.

TABLE 6
PCR Master Mix components utilized in
PCR prior to 16S rRNA gene sequencing.
AccuPrime ™ Taq DNA Polymerase System

10X AccuPrime ™ PCR Buffer II (500 μL) containing:
200 mM Tris-HCl (pH 8.4)
500 mM KCl
15 mM MgCl₂
2 mM dGTP
2 mM dATP
2 mM dTTP
2 mM dCTP
Thermostable AccuPrime ™ protein
10% glycerol
50 mM Magnesium Chloride (500 μl)

TABLE 7
Cycling process utilized in PCR prior
to 16S rRNA gene sequencing.

| Run Stage | Temperature | Time | |
|---|---|---|---|
| Step 1 | 95° C. | 3 | minutes |
| Step 2: (25 cycles) | 95° C. | 30 | seconds |
| Step 3 | 55° C. | 30 | seconds |
| Step 4 | 72° C. | 30 | seconds |
| Step 5 | 72° C. | 5 | minutes |

| | |
|---|---|
| Step 6: Hold | 4° C. |

The total reaction volume used was 20 μL with 20 ng of sample DNA.

TABLE 8
PCR FastMix components utilized in RT-PCR.
PerfecCTa SYBR ® Green FastMix

Reaction buffer with optimized concentrations of molecular-grade MgCl2, dATP, dCTP,
dGTP, and dTTP
AccuStart II Taq DNA Polymerase
SYBR Green I dye
Proprietary enzyme stabilizers and performance-enhancing additives

TABLE 9
Cycling methods for RT-PCR, including run stage
with associated temperature and duration

| Run Stage | Temperature | Time | |
|---|---|---|---|
| Hold | 50° C. | 2 | minutes |
| | 95° C. | 10 | minutes |
| PCR (40 cycles) | 95° C. | 15 | seconds |
| | 60° C. | 10 | minutes |
| Continuous melt curve | 95° C. | 15 | seconds |
| | 60° C. | 15 | seconds |
| Dissociation | 95° C. | 15 | seconds |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for diagnosing infectious bovine keratoconjunctivitis (IBK) or squamous cell carcinoma (OSCC) in cattle, comprising
a) assaying a conjunctival swab from the cattle by quantitative PCR (qPCR) using forward and reverse primers specific for a panel of targets comprising Bovine GAPDH, Moraxella bovis, Moraxella bovoculi, Mycoplasma, Staphylococcus, Pasteurellaceae, Prevotellaceae, Weeksellaceae, Universal Bacteria, and Escherichia coli;
b) assaying a known standard of E. coli by quantitative PCR (qPCR) using forward and reverse primers specific for Universal Bacteria and Escherichia coli;
c) calculating average Cycle Threshold (C_{T}) value for each target;
d) normalizing the cattle average C_{T} values using the known standard of E. coli average C_{T} values;
e) comparing the normalized C_{T} values to control values to predict the presence of IBK or OSCC in the cattle.

2. The method of claim 1, wherein the forward and reverse primers for Bovine GAPDH comprise the nucleic acid sequences SEQ ID NO:1 and 2, respectively.

3. The method of claim 1, wherein the forward and reverse primers for Moraxella bovis comprise the nucleic acid sequences SEQ ID NO:3 and 4, respectively.

4. The method of claim 1, wherein the forward and reverse primers for Moraxella bovoculi comprise the nucleic acid sequences SEQ ID NO:5 and 6, respectively.

5. The method of claim 1, wherein the forward and reverse primers for Mycoplasma comprise the nucleic acid sequences SEQ ID NO:7 and 8, respectively.

6. The method of claim 1, wherein the forward and reverse primers for Staphylococcus comprise the nucleic acid sequences SEQ ID NO:9 and 10, respectively.

7. The method of claim 1, wherein the forward and reverse primers for Pasteurellaceae comprise the nucleic acid sequences SEQ ID NO:11 and 12, respectively.

8. The method of claim 1, wherein the forward and reverse primers for Prevotellaceae comprise the nucleic acid sequences SEQ ID NO:13 and 14, respectively.

9. The method of claim 1, wherein the forward and reverse primers for Weeksellaceae comprise the nucleic acid sequences SEQ ID NO:15 and 16, respectively.

10. The method of claim 1, wherein the forward and reverse primers for Universal Bacteria comprise the nucleic acid sequences SEQ ID NO:17 and 18, respectively.

11. The method of claim 1, wherein the forward and reverse primers for Escherichia coli comprise the nucleic acid sequences SEQ ID NO:19 and 20, respectively.

12. The method of claim 1, wherein step e) comprises the use of a data analysis algorithm to weight the C_{T} values according to the coefficients of Table 4.

13. The method of claim 1, wherein the method predicts the presence of IBK or OSCC, the method further comprising treating the cattle for IBK or OSCC.


SEQUENCE LISTING (20 sequences)

SEQ ID NO: 1 - DNA, 20 bp, unidentified
  source 1..20: mol_type = unassigned DNA
cctggagaaacctgccaagt

SEQ ID NO: 2 - DNA, 21 bp, unidentified
  source 1..21: mol_type = unassigned DNA
gccaaattcattgtcgtacca

SEQ ID NO: 3 - DNA, 18 bp, unidentified
  source 1..18: mol_type = unassigned DNA
ggtgacgaccgcttgttt

SEQ ID NO: 4 - DNA, 20 bp, unidentified
  source 1..20: mol_type = unassigned DNA
atcatcgccttcatctccag

SEQ ID NO: 5 - DNA, 24 bp, unidentified
  source 1..24: mol_type = unassigned DNA
ggtgatatttatcatgaagttgtg

SEQ ID NO: 6 - DNA, 27 bp, unidentified
  source 1..27: mol_type = unassigned DNA
tctcaattcataatcacgatactcaag

SEQ ID NO: 7 - DNA, 27 bp, unidentified
  source 1..27: mol_type = unassigned DNA
tgcaccatctgtcactctgttaacctc

SEQ ID NO: 8 - DNA, 22 bp, unidentified
  source 1..22: mol_type = unassigned DNA
actcctacgggaggcagcagta

SEQ ID NO: 9 - DNA, 25 bp, unidentified
  source 1..25: mol_type = unassigned DNA
ggccgtgttgaacgtggtcaaatca

SEQ ID NO: 10 - DNA, 23 bp, unidentified
  source 1..23: mol_type = unassigned DNA
accatttcagtaccttctggtaa

SEQ ID NO: 11 - DNA, 17 bp, unidentified
  source 1..17: mol_type = unassigned DNA
cataagatgagcccaag

SEQ ID NO: 12 - DNA, 18 bp, unidentified
  source 1..18: mol_type = unassigned DNA
gtcagtacattcccaagg

SEQ ID NO: 13 - DNA, 21 bp, unidentified
  source 1..21: mol_type = unassigned DNA
ggttctgagaggaaggtcccc

SEQ ID NO: 14 - DNA, 20 bp, unidentified
  source 1..20: mol_type = unassigned DNA
tcctgcacgctacttggctg

SEQ ID NO: 15 - DNA, 17 bp, unidentified
  source 1..17: mol_type = unassigned DNA
atccagccatcccgcgt

SEQ ID NO: 16 - DNA, 19 bp, unidentified
  source 1..19: mol_type = unassigned DNA
ctgctggcacggagttagc

SEQ ID NO: 17 - DNA, 18 bp, unidentified
  source 1..18: mol_type = unassigned DNA
cctacgggaggcagcagt

SEQ ID NO: 18 - DNA, 17 bp, unidentified
  source 1..17: mol_type = unassigned DNA
attaccgcggctgctgg

SEQ ID NO: 19 - DNA, 20 bp, unidentified
  source 1..20: mol_type = unassigned DNA
ccgatacgctgccaatcagt

SEQ ID NO: 20 - DNA, 20 bp, unidentified
  source 1..20: mol_type = unassigned DNA
acgcagaccgtaggccagat